(19) United States
(12) Patent Application Publication
(10) Pub. No.: US 2008/0261862 A1
(43) Pub. Date: Oct. 23, 2008

(54) Materials and Methods for Modulating Signalling by Alpha-V Integring Molecules

(76) Inventors: William Cushley, Lanarkshire (GB); Gillian Borland, Lanarkshire (GB); Bradford Ozanne, Lanarkshire (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/573,263

(22) PCT Filed: Aug. 5, 2005
(86) PCT No.: PCT/GB2005/003097

§ 371 Date: Feb. 14, 2008

(30) Foreign Application Priority Data
Aug. 5, 2004 (GB) 0417490.0

(51) Int. Cl. A61K 38/02; A61P 35/00; A61P 29/00; A61K 31/70; G01N 33/53; C07K 1/14; C07K 7/00; C12N 15/12; C12N 5/10; A01K 67/027; C12N 15/68

(52) U.S. Cl. 514/2; 514/44; 536/23.1; 435/7.1; 800/3; 530/344; 530/300; 530/330; 530/329; 530/328; 435/352; 800/14; 435/320.1

(57) ABSTRACT

It is disclosed that the αvβ5 integrin mediates the proliferative signal provided by CD23 to pre-B cells. The region of CD23 which interacts with αvβ5 has been defined, and found to interact with a site on the integrin distinct from that which binds RGD. The invention provides methods for disrupting the interaction between CD23 and αvβ5 and methods of screening for chemical entities capable of disrupting this interaction.

### FIELD OF THE INVENTION

The invention relates to integrins, and in particular to the interaction of CD23 with αv integrins. The present invention establishes for the first time that αvβ5 mediates the proliferative signal provided by CD23 to pre-B cells, and defines the region of CD23 which interacts with αvβ5.

### BACKGROUND TO THE INVENTION

CD23 is a 45 kDa type II transmembrane glycoprotein expressed by haematopoietic cells that functions as the low affinity receptor for IgE¹⁻³. As a membrane protein, CD23 negatively regulates IgE production and IgE-dependent antigen focussing and presentation by B lymphocytes^{4,5}. CD23 exists as monomeric and trimeric structures at the plasma membrane, with the latter having a higher affinity for IgE⁶⁷. CD23 is cleaved by membrane-associated metalloproteases^{8,9} to yield a range of soluble CD23 species (sCD23) of molecular weights 37 kDa, 33 kDa, 29 kDa, 25 kDa and 16 kDa. All sCD23 molecules retain the capacity to bind IgE and exhibit pleiotropic cytokine-like activities^{2,3}. The region of sCD23 responsible for IgE binding overlaps with, but is distinct from, that required for cytokine activity¹⁰. Soluble CD23 is also oligomeric, and cross-linking studies suggest that trimeric, and possibly hexameric, forms of sCD23 are biologically active¹¹. Functionally, sCD23 promotes differentiation of monocyte and early thymocyte precursors^{12,13}, in association with IL-1α. In germinal centres, sCD23 not only inhibits apoptosis of centrocytes¹⁴ but also promotes differentiation of surviving centrocytes to plasmablasts. Soluble CD23 also inhibits apoptosis in pre-B cell lines¹⁵, and drives synthesis of TNFα and IL-1α by monocytes^{16,17}. Plasma levels of sCD23 correlate with disease status in a range of pathological conditions¹⁸, including a range of allergic and atopic disorders, following certain parasitic and viral infections¹⁹, and also the inflammatory diseases, Sjögrens Syndrome, systemic lupus erythematosus²⁰, thyroiditis and rheumatoid arthritis^{21 22}. Strikingly high levels of sCD23 are found in the plasma of B-chronic lymphoblastic leukaemia (B-CLL) patients, where the absolute levels of sCD23 and the kinetics of its elevation have incisive prognostic value²³.

Human CD23 binds to a range of cell surface receptors. As a membrane-associated protein, CD23 associates with CD21²⁴ and promotes homotypic adhesion²⁵ between activated B cells. Certain anti-CD21 MAbs mimic the capacity of sCD23 to rescue centrocytes from apoptosis²⁶, demonstrating that CD21 mediates the action of CD23 in centrocytes. CD23 binding to CD21 requires both protein-protein and protein-carbohydrate interactions²⁷, indicating that the lectin head domain of CD23 mediates CD21 binding. In monocytes, sCD23 binds the α2 integrins, CD11b-CD18 (αMβ2) and CD11c-CD18 (αXβ2)¹⁶, and to the αvβ3 vitronectin receptor (VnR) isoform¹⁷ promoting pro-inflammatory cytokine synthesis. The protein domains responsible for the CD23-integrin interactions are not defined. Murine CD23 binds to CD21, and also to the CD11b-CD18 α2 integrin in murine macrophages 28 where production of IL-6 is stimulated. It remains to be formally demonstrated whether murine CD23 binds αv integrins.

Vitronectin receptors (VnRs) comprise the αv integrin subunit in non-covalent association with one of five β subunits, β1, β3, β5, β6 and β8²⁹, and have important roles in cell attachment to, and migration on, substrates, rescue from apoptosis, and angiogenesis³⁰⁻³³. Monocytes utilise αvβ3 and αvβ5 to phagocytose apoptotic cells 34 and, when ligated by CD23, to promote pro-inflammatory cytokine synthesis^{16,17}. VnRs function to bind extracellular matrix proteins including vitronectin (Vn) and fibronectin (Fn), by recognition of arg-gly-asp (RGD) motifs³⁵, and the structural biology of RGD binding by the αvβ3 integrin is now well understood. Briefly, RGD peptides are secured by structures on the β-propellor domain of the αv subunit and by liganding residues in the βA domain of the β3 subunit^{36,37}. Binding of RGD ligands causes significant conformational change in the integrin itself and the ligand. However, there are data to suggest that integrins bind target proteins via motifs other than RGD³⁵. In particular, αvβ5 binds to a basic domain on the HIV Tat protein³⁸. The contribution of the basic domain-binding site to integrin function remains obscure, but the greater affinity of αvβ5 for the Tat basic domain compared to the equivalent Vn domain suggests that ligands other than Vn interact with the αvβ5 basic domain-binding site and have distinct signalling functions.

### SUMMARY OF THE INVENTION

The present inventors have now found that rescue of human B cell precursors from apoptosis by sCD23¹⁵ is mediated via the αvβ5 VnR isoform. They have also found that the site on the αvβ5 integrin responsible for binding CD23 is distinct from the RGD-binding pocket, and have mapped the residues on CD23 which mediate the interaction.

In a first aspect the invention provides a method of inhibiting binding between an αv integrin and CD23, the method comprising contacting the αv integrin with a peptide capable of binding to αvβ5, the peptide comprising the motif X1X2X3, wherein at least one of X1, X2 and X3 is a residue carrying at least a partial positive charge at physiological pH, and one of X1 and X3 is C.

The methods of the invention may be performed in vitro, ex vivo, or in vivo. They include cellular and molecular assays carried out in vitro, as well as methods of modulating cellular effects of αv/CD23 interaction in vitro and in vivo, and other applications of the findings described in this specification.

In the various methods described, the αv integrin and the CD23 protein may each be in any suitable physical form. For example, they may be cell-associated, immobilised on a solid phase, or in free solution.

By “cell-associated” is meant covalently or non-covalently bound to the exterior surface of a cell's plasma membrane, or extending through the plasma membrane as an integral membrane protein.

The CD23 may be any of the known soluble CD23 species (including the 37, 33, 29, 25 and 16 kDa forms) as well as the integral membrane form of the protein. CD23 exists in at least two isoforms in humans and also in mice. These isoforms differ only in their intracellular sequence; the extracellular domain responsible for interaction with integrins does not vary. Therefore it is believed that the various methods of the invention may be applied with any isoform of CD23.

It is believed that the peptides described in this specification are capable of inhibiting interaction between CD23 and αv integrins other than αvβ5. Thus the αv integrin may be any αv integrin capable of binding to CD23, e.g. αvβ3, αvβ5, αvβ6 or αvβ8.

In certain embodiments, the interaction involves a cell expressing the αv integrin and soluble or cell-associated CD23. Thus the methods of the invention may be used to inhibit one or more of the cellular effects mediated by CD23 interaction with cell-associated αv integrins. For example, CD23 can stimulate cell proliferation and/or promote cell survival, e.g. in pre-B cells, which enter apoptosis without stimulation by CD23, and in certain cancer cells, such as acute lymphoblastic leukaemia (ALL) cells. Myeloma (multiple myeloma) cells have high levels of αvβ5/αvβ3, and may also receive proliferative or anti-apoptotic signals from CD23. CD23 can also stimulate secretion of inflammatory cytokines (including IL-1α and TNF-α) by monocytic cells such as monocytes and macrophages.

Thus the invention provides a method of inhibiting CD23-dependent proliferation or survival of a cell, comprising contacting said cell with a peptide capable of binding to αvβ5, the peptide comprising the motif X1X2X3, wherein at least one of X1, X2 and X3 is a residue carrying at least a partial positive charge at physiological pH, and one of X1 and X3 is C.

The cell may be a pre-B cell, or a cancer cell such as an ALL cell, particularly an ALL cell of the B cell lineage, or a myeloma cell, such as a cell from multiple myeloma.

The invention therefore provides a method of treating cancer in a subject, comprising administering to the subject an effective amount of (i) a peptide capable of binding to αvβ5, the peptide comprising the motif X1X2X3, wherein at least one of X1, X2 and X3 is a residue carrying at least a partial positive charge at physiological pH, and one of X1 and X3 is C, or (ii) a nucleic acid encoding said peptide.

The invention further provides the use of a peptide as described herein, or a nucleic acid encoding the same, in the preparation of a medicament for the treatment of cancer.

The invention further provides a method of inhibiting pro-inflammatory cytokine secretion from a monocytic cell comprising contacting the cell with a peptide capable of binding to αvβ5, the peptide comprising the motif X1X2X3, wherein at least one of X1, X2 and X3 is a residue carrying at least a partial positive charge at physiological pH, and one of X1 and X3 is C.

Suitable monocytic cells include monocytes and macrophages. The cytokine secretion which it is intended to inhibit may be triggered by CD23 binding to an αv integrin, including αvβ3, αvβ5, αvβ6 and αvβ8. The cytokine in question may be one or more of IL-1 (e.g. IL-1α), TNF-α, IL-6, IL-8, IL-12 and IFN-γ.

Typically, the cell will be in an activated, pro-inflammatory state as a result of stimuli other than CD23/αv interactions. This interaction may enhance pro-inflammatory cytokine secretion, but is not generally sufficient in isolation to induce such secretion.

The method may be applied in vivo or in vitro, and therefore the invention also includes a method of treating an inflammatory disorder in a subject, comprising administering to the subject an effective amount of (i) a peptide capable of binding to αvβ5, the peptide comprising the motif X1X2X3, wherein at least one of X1, X2 and X3 is a residue carrying at least a partial positive charge at physiological pH, and one of X1 and X3 is C, or (ii) a nucleic acid encoding said peptide.

The invention further provides the use of a peptide as described herein in the preparation of a medicament for the treatment of an inflammatory disorder.

Any inflammatory disorder in which TNF-α, IL-1 (e.g. IL-1α), IL-6, IL-8, IL-12, IFN-γ or other monocyte-derived pro-inflammatory cytokine is implicated in pathogenesis may be treated in this way. For example, rheumatoid arthritis has been shown to be treatable using anti-TNF-α. Other suitable conditions include Sjogren's Syndrome, systemic lupus erythaematosus (SLE), sarcoidosis, endometriosis, thyroiditis and atherosclerosis. Particularly suitable conditions are those in which binding between CD23 and an αv integrin triggers or contributes to the secretion of the pro-inflamatory cytokine.

In a further aspect the invention provides a method of screening for a substance capable of inhibiting binding between an αv integrin and CD23, the method comprising

(i) contacting the αv integrin with a test substance,
(ii) contacting the αv integrin with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(iii) determining binding of the peptide to the αv integrin.

As described above, the αv integrin may be in any suitable physical form. Conveniently, though, the integrin may be associated with a cell or immobilised on a solid phase, e.g. a bead or surface of a microtitre plate. Alternatively, although possibly less conveniently, the peptide may be immobilised on a solid phase.

The test substance may be selected or rejected depending on its effect on peptide binding to the αv integrin. Typically, those which inhibit or reduce peptide binding to αv will be selected as potential inhibitors of the αv/CD23 interaction.

The screening assays described may be applied to panels of hundreds or thousands of test substances, e.g. in high-throughput assays. However, it will be appreciated that the methods may also be applied to just one or a few test substances, in order to determine their effect. The test substances may already be known or suspected to affect binding between CD23 and an αv integrin. The term “screening” should be construed accordingly.

The peptide may be labelled to facilitate detection of the interaction with the αv integrin, as described in more detail below.

The skilled person is aware of numerous suitable formats for such screening assays, and will be capable of selecting an appropriate format depending on their individual requirements.

Typically, the αv integrin is contacted sequentially (in any order), or simultaneously, with the test substance and the peptide. The extent of peptide binding to the integrin is then determined either by directly or indirectly labelling the peptide, and detecting the amount of label associated with the integrin. For example, the peptide may be directly or indirectly labelled with radioactive, fluorescent, chemiluminescent or enzyme labels (such as alkaline phosphatase or horseradish peroxidase) so that they can be detected using techniques well known in the art.

Indirect labelling may be achieved by coupling the label to a member of a specific binding pair, which is capable of binding to a complementary member of the specific binding pair associated with the peptide or the peptide-integrin complex. Examples of specific binding pairs include antibodies and their cognate epitopes, avidin/streptavidin and biotin, lectins and carbohydrates, etc.

Thus the complex may be detected using a labelled antibody which is capable of binding to the peptide itself, to a moiety associated (e.g. linked covalently or non-covalently) with the peptide, or to the peptide-integrin complex. For example, the peptide may be linked (e.g. as a fusion protein) with a peptide or protein comprising an epitope for the antibody. Alternatively, the antibody may be specific for the peptide itself or for an epitope created by binding of the peptide to the integrin.

Alternatively the peptide may be associated with biotin or another similar member of a specific binding pair, and may be detected using labelled avidin/streptavidin or the appropriate complementary member of the specific binding pair.

Radioactive labels can be detected using a scintillation counter or other radiation counting device, fluorescent labels using a laser and confocal microscope, and enzyme labels by the action of an enzyme label on a substrate, typically to produce a spectrophotometrically detectable colour change.

Further techniques which may be used include flow cytometry and immunohistochemistry, which may be appropriate if the peptide or integrin is cell-associated. Alternatively, peptide-integrin binding may be determined directly using surface plasmon resonance.

The signal generation methods described in The Immunoassay Handbook (Second Edition) Ed D Wild published by the Nature Publishing Group (2001) may also be applied. Of particular application are those homogeneous systems described in Chapter 11 (E. F. Ullman). Scintillation Proximity Assay (SPA) (with for example a weak alpha or beta-emitter and a fluorophore) and Enzyme Channelling (with for example glucose oxidase and peroxidase) provide particularly attractive systems for use in the methods described. In these methods the peptide and integrin may be labelled with complementary components of the detection system such that when they aggregate the two components are brought closely enough together to produce a detectable signal but when not complexed no such association occurs and thus no signal is produced.

The peptides which bind to αvβ5 are also capable of interacting with other αv integrins comprising other β chains, e.g. β3, 6 and 8. Surprisingly, the present inventors have found that the peptides appear to interact primarily with the β chain, rather than with the common α chain. Thus the screening methods described above may utilise an isolated β chain, or an extracellular domain thereof, instead of a complete αv integrin. Thus the screening method may comprise contacting an isolated integrin β chain with the test substance and the peptide, and determining binding of the peptide to the isolated β chain. Preferably the β chain is β5, although others may be used, including β1, 3, 6 or 8. β1 may be used even though CD23 does not appear to bind to αvβ1. However it is less preferred because the results obtained may be less physiologically relevant than those obtained with other β chains. It is not known why the peptides will bind to β1 when the full CD23 molecule does not bind βvβ1. It is possible that a CD23 binding site exists on all of these β chains, and for some reason is sterically blocked in the αvβ1 complex but not in other αv integrins.

Conveniently, isolated β chain (e.g. recombinantly-expressed β chain) or an extracellular domain thereof may be immobilised on a solid phase for use in such assays, as described above.

Whether the screening methods are carried out using an αvβ complex, or an isolated β chain, they may further comprise contacting a cell expressing an αv integrin with said test substance and determining the effect of the test substance on the cell. This may involve determining whether the test substance affects the apoptotic state of the cell (e.g. induces the cell to enter apoptosis), determining the effect of the test substance on proliferation of the cell, or determining the effect of the test substance on cytokine expression and/or secretion by the cell, typically in response to a suitable stimulus.

Thus the invention provides a method of screening for a substance having anti-cancer activity, comprising, having performed a method of screening for a substance capable of inhibiting the interaction between an αv integrin and CD23 as described above, contacting a cancer cell with said test substance and determining the effect of the substance on proliferation and/or apoptosis of the cell.

The invention further provides a method of screening for a substance having anti-inflammatory activity, comprising, having performed a method of screening for a substance capable of inhibiting the interaction between an αv integrin and CD23 as described above, contacting a monocytic cell with said test substance and determining the effect of the substance on inflammatory cytokine expression and/or secretion by the cell. The method may comprise the step of administering a suitable stimulus to the cell which, in the absence of the test substance, would be expected to provoke inflammatory cytokine expression and/or secretion. The method may involve determining expression and/or secretion of, for, example, TNF-α, IL-1 (e.g. IL-1α), IL-6, IL-8, IL-12, IFN-γ or any other suitable pro-inflammatory cytokine.

While both of the above screening methods may be performed in vitro, they may also be performed in vivo, e.g. in experimental animal models, in order to examine the effect of the test substance on disease progression. So, to determine the effect of the test substance on cancer cells, the substance may be administered to a test animal having a suitable cancer, e.g. inoculated with a pre-B cell cancer cell expressing αv, such as SMS-SB (refs. 15, 39; see also Smith et al., (1981) J. Immunol. 126(2), p. 596). To determine the effect of the test substance on inflammatory disease, the substance may be administered to an animal model for that disease, e.g. a model for rheumatoid arthritis, such as the murine collagen-induced arthritis model. Of course, when the assays are performed in vitro, they may use cells from such animal models.

When the methods are performed in vivo, the animal is typically sacrificed as part of the assay. This may be necessary to obtain tissue samples to study the effect of the test substance on disease.

The animal may be a rodent (e.g. a mouse or rat), or any other suitable laboratory animal such as a rabbit, guinea pig, cat, dog, etc.

Preferably the methods are performed using rodent cells or transgenic rodents capable of expressing a CD23 protein having R at a position corresponding to R172 of human CD23. Such cells and transgenic rodents are described in more detail below.

In a further aspect the invention provides a method of stimulating proliferation or inhibiting apoptosis of a cell expressing an αv integrin, the method comprising contacting the cell with an αvβ5 agonist peptide, the peptide comprising the motif X1X2X3, wherein at least one of X1, X2 and X3 is a residue carrying at least a partial positive charge at physiological pH, and one of X1 and X3 is C

The cell preferably expresses αvβ5. It may, for example, be a pre-B cell.

The method may be performed in vitro, in vivo, or ex vivo. For example, the method may be used to repopulate an individual's B cell compartment after depletion thereof, e.g. by irradiation or drug treatment. Cells may be treated in vitro and administered to the subject, or alternatively the peptide may be administered directly to the subject, e.g. to the bone marrow.

In a further aspect, the invention provides a method of determining expression of an αv integrin by a cell, the method comprising:

(i) contacting the cell with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(ii) determining binding of said peptide to said cell.

The method will typically comprise the step of correlating the result with the level of expression of αv. It will be apparent that the higher the level of binding observed, the higher the level of expression of the αv integrin by the cell.

The peptide may be labelled to facilitate detection.

The method may comprise a control or comparison step, involving contacting a control cell having a known expression level of αv with the peptide. The control cell may be known not to express αv.

The method may comprise the further step of determining the expression of an associated integrin β chain, e.g. β1, 3, 5, 6 or 8.

αvβ5 is expressed at particularly high levels on ALL cells. Thus the invention provides a method of screening for the presence of an ALL cell in a sample comprising blood cells, the method comprising

(i) contacting the sample with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(ii) determining binding of said peptide to said blood cells.

The method may comprise a control or comparison step of contacting a normal blood sample (e.g. known or suspected not to contain ALL cells) with the peptide and determining the level of binding. This will provide a reference level against which the level of binding in the test sample can be compared. Additionally or alternatively a positive control sample, known to contain ALL cells, may be used.

The method is particularly useful for the detection of ALL cells of the B cell lineage. Normal peripheral B cells are thought not to express significant levels of αvβ5, so the presence of αvβ5 on peripheral B cells is a strong indicator of the presence of ALL cells.

Thus the method may comprise contacting the sample with a B cell-specific binding agent, i.e. substance capable of binding specifically to B cells, e.g. to a B cell-specific marker, such as CD19 or CD20. Antibodies are particularly preferred. This step may be performed before contacting the sample with the peptide (e.g. in order to separate the B cells from other cells in the sample), concurrently with, or after the peptide.

The B cell-specific agent may also be labelled; typically it will carry a different label to that carried by the peptide, to allow independent detection.

Suitable techniques and labels are described above in relation to assays for identifying substances capable of disrupting the interaction between αv integrins and CD23.

The skilled person is aware of numerous suitable formats for such diagnostic assays, and will be capable of selecting an appropriate format depending on their individual requirements.

In a further aspect the invention provides a method of isolating an αv integrin from a sample comprising contacting said sample with a peptide capable of binding to αvβ5, the peptide comprising the motif X1X2X3 wherein at least one of X1, X2 and X3 is a residue carrying at least a partial positive charge at physiological pH, and one of X1 and X3 is C.

The sample may be any sample known or suspected to contain an αv integrin, such as a cell lysate or a fraction thereof.

The peptide may be immobilised on a solid phase, or associated with a member of a specific binding pair, in order to facilitate purification of the peptide-integrin complex from the sample.

As described above, examples of specific binding pairs include antibodies and their cognate epitopes, avidin/streptavidin and biotin, lectins and carbohydrates, etc.

Thus the method may include the step of contacting the sample with a member of a specific binding pair, capable of binding to a complementary member of the specific binding pair associated with the peptide or the peptide-integrin complex. For example the member of the specific binding pair may be an antibody which is capable of binding to the peptide, to a moiety associated covalently or non-covalently with the peptide, or to the peptide-integrin complex itself. For example, the peptide may be associated (e.g. as a fusion protein) with a peptide or protein comprising an epitope for the antibody. The member of the specific binding pair may be associated with a solid phase, e.g. a bead or magnetic particle, to facilitate isolation of the complex by centrifugation or application of a magnetic field. Alternatively it may be multivalent, to enable precipitation of the complex from solution.

In a further aspect the invention provides a method of inhibiting CD23-dependent proliferation or survival of a cell, comprising contacting said cell with a substance capable of inhibiting the interaction between CD23 and αvβ5.

The cell may be a pre-B cell, or a cancer cell such as a myeloma cell or an ALL cell, particularly an ALL cell of the B cell lineage.

As described above, the substance may be a peptide capable of binding to αvβ5, the peptide comprising the motif X1X2X3 wherein at least one of X1, X2 and X3 is a residue carrying at least a partial positive charge at physiological pH, and one of X1 and X3 is C.

In a further aspect the invention provides a method of screening for a substance capable of inhibiting CD23-dependent proliferation or survival of a cell, the method comprising

(i) contacting an αv integrin with a test substance,
(ii) contacting the αv integrin with CD23, and
(iii) determining binding of CD23 to the αv integrin.

Further features of this aspect of the invention are as described above in relation to methods of screening for substances capable of disrupting the interaction between αv integrins and CD23, mutatis mutandis. Thus, part or substantially all of any one of the known forms of CD23 may be used in place of the peptides described in that aspect of the invention. An isolated β chain (or extracellular domain thereof) may be used in place of a complete αv integrin.

In those aspects of the invention described above which relate to a peptide capable of binding to αvβ5, the peptide comprises a motif X1X2X3, wherein at least one of X1, X2 and X3 is a residue carrying at least a partial positive charge at physiological pH, and one of X1 and X3 is C.

The motif preferably comprises only one C residue. Either or both of the remaining residues may carry at least a partial positive charge at physiological pH.

Preferably, the residue or residues carrying at least a partial positive charge at physiological pH is K or R. Other residues may be suitable, including H, and non-naturally-occurring residues which carry a whole or partial positive charge at physiological pH. In this context, physiological pH is considered to be pH 7.4. It will be understood that a single residue cannot carry a partial positive charge. This term instead refers to the average charge on the relevant residue over the whole population of peptides in a given system. This will be between 0 and 1 if the pK of the residue is close to 7, e.g. between about 6 and about 9.5.

Preferably, any of X1, X2 and X3 which is neither C nor a residue carrying at least a partial positive charge is a neutral residue, i.e. a residue which does not carry a charge at physiological pH. Such amino acids include Q, N, A, G, S, T, I, L, M, F, P, W, Y, V. In preferred embodiments, such amino acids are Q, N, A, G, S, T, V, L or I, still more preferably A, Q, S or G. It will be appreciated that the conventional one letter codes for amino acids are used throughout this specification, and their use should be construed accordingly.

In certain embodiments the peptides may comprise the motif X1X2X3, wherein one of X1 or X3 is C, the other of X1 and X3 is Q, R, K, H, A, G or S, and X2 is K, R, Q, H, A, G or S, provided that one of X1, X2 and X3 is K, R or H. Preferably one of X1, X2 and X3 is K or R.

In some embodiments, one of X1 and X3 is C, the other of X1 and X3 is Q, R or A, and X2 is K or A, provided that one of X1, X2 and X3 is K or R.

The peptide may comprises the motif XaX1X2X3 or X1X2X3Xa, wherein Xa is Q, K or R, preferably Q.

Preferred sequences for the motif X1X2X3 include RKC, KKC, QKC, AKC, RAC and CKR.

The peptides may be up to 5, 6, 7, 8, 9, 10, 15, 20, 25, 30, 35, 40, 45 or 50 amino acids in length, or more.

The peptide may comprise one or more repeats of a CD23 sequence of up to 20 amino acids, the CD23 sequence comprising the motif X1X2X3 as described above, and further having at least 60%, 70%, 80%, 85%, 90% or 95% sequence identity with the corresponding portion of amino acids 155 to 191 of SEQ ID NO: 1. The CD23 sequence may have 100% identity with the corresponding portion of amino acids 155 to 191 of SEQ ID NO: 1.

```
SEQ ID NO: 1 is the sequence of human CD23a having GenBank accession
numbers AAH62591.1; GI:33511828:
    1 MEEGQYSEIE ELPRRRCCRR GTQIVLLGLV TAALWAGLLT LLLLWHWDTT QSLKQLEERA
  61 ARNVSQVSKN LESHHGDQMA QKSQSTQISQ ELEELRAEQQ RLKSQDLELS WNLNGLQADL
121 SSFKSQELNE RNEASDLLER LREEVTKLRM ELQVSSGFVC NTCPEKWINF QRKCYYFGKG
181 TKQWVHARYA CDDMEGQLVS IHSPEEQDFL TKHASHTGSW IGLRNLDLKG EFIWVDGSHV
241 DYSNWAPGEP TSRSQGEDCV MMRGSGRWND AFCDRKLGAW VCDRLATCTP PASEGSAESM
301 GPDSRPDPDG RLPTPSAPLH S
```

Amino acids 155 to 191 are underlined. The RKC motif, at amino acids 172 to 174, is shown in bold. The extracellular domain is believed to begin at H46.

Alignments and degrees of sequence identity may be determined, for example, using the program BLAST (provided by the National Center for Biotechnology Information) using default parameters. The peptide sequence is aligned with the relevant protein sequence, and the degree of identity along the overlap between the two sequences is determined.

Preferably the CD23 sequence is at least 5 amino acids in length. Thus it may be 5, 6, 7, 8, 9, 10, 11, 12, 13, 14, 15, 16, 17, 18, 19 or 20 amino acids in length.

The peptide may comprise any of the sequences

QRKC
FQRKC
NFQRKC
INFQRKC
WINFQRKC
KWINFQRKC
RKCY
RKCYY
RKCYYF
RKCYYFG
RKCYYFGK
RKCYYFGKG
QRKCYY
FQRKCYY
FQRKCYYF
FQRKCYYFG
NFQRKCY
INFQRKCY
INFQRKCYY

Alternatively the peptide may comprise an equivalent sequence from a non-human CD23 protein, e.g. a mammalian CD23 protein, such as a murine CD23 protein. The equivalent sequence may be determined by aligning the human sequence with the non-human sequence, for example using the program BLAST (provided by the National Center for Biotechnology Information) using default parameters. Those portions of bovine, murine, equine and rat CD23 which correspond to residues 155 to 191 of human CD23 are shown below:

| | |
|---|---|
| 155-191 | |
| Human | SSGFVCNTCPEKWINFQRKCYYFGKGTKQWVHARYA |
| Bovine | ANGSVCNTCPEAWIYFQKKCYYFGEGAKKWIQARYA |
| Horse | SNGSTCNTCPDDWVHFQKKCYYFGEGPKRWIQARFA |
| Mouse | SKGTACNICPKNWLHFQQKCYYFGKGSKQWIQARFA |
| Rat | SKGTACNVCPKDWLHFQQKCYYFGEGSKQWIQAKFT |

It may be desirable that the peptide is not an agonist of αvβ5 in its own right. In various of the aspects described above, it will be desirable that the peptide is an antagonist, that is to say it inhibits the ability of CD23 to induce signalling through the relevant αv integrin.

Without wishing to be bound by any particular theory, peptides which do not show agonist activity preferably comprise at least one further amino acid, and preferably at least two further amino acids, N-terminal and C-terminal of the X1X2X3 motif.

In other embodiments it may be desirable to use a peptide that is an agonist of αvβ5. Examples include RKCYYFGKG and KWINFQRKC.

The peptide may be cyclic. For example, it may comprise two further C residues in addition to that in the X1X2X3 motif which together form an intermolecular disulphide bond.

The peptides described preferably do not competitively inhibit binding between αv integrins and αv ligands containing RGD motifs (e.g. fibronectin and vitronectin). Without wishing to be bound by any particular theory, it is believed that they interact with a site on αv integrins distinct from that which binds ligands containing RGD motifs.

The present invention further provides a peptide or polypeptide comprising a CD23 sequence of up to (but not exceeding) 20 amino acids,

the CD23 sequence comprising the motif X1X2X3, wherein at least one of X1, X2 and X3 is a residue carrying at least a partial positive charge at physiological pH, and one of X1 and X3 is C,
the CD23 sequence further having at least 50%, 60%, 70% or 80% sequence identity with the corresponding portion of amino acids 155 to 191 of SEQ ID NO: 1.

The CD23 sequence may be a naturally occurring sequence of a mammalian CD23 molecule, e.g. human, murine, rat, equine or bovine CD23, as shown above, or may comprise one or more amino acid insertions, deletions or substitutions, as long as it retains the overall degree of sequence identity with the overlapping sequence of SEQ ID NO: 1.

Preferably the CD23 sequence of the peptide or polypeptide has at least 85%, 90%, 95% or 100% identity to the corresponding region of SEQ ID NO: 1.

Preferably the CD23 sequence is at least 5 amino acids in length. Thus it may be 5, 6, 7, 8, 9, 10, 11, 12, 13, 14, 15, 16, 17, 18, 19 or 20 amino acids in length.

The peptide or polypeptide may contain more than one repeat of the CD23 sequence. For example, it may contain two repeats separated by a flexible linker. However, the peptide or polypeptide preferably does not contain any sequences of more than 5 contiguous amino acids from any other part of a CD23 molecule other than the CD23 sequence specified.

Preferably the remainder of the polypeptide or peptide does not comprise more than 5 contiguous amino acids from a CD23 sequence, and/or displays less than 20% sequence identity with a CD23 molecule.

Preferred features of the peptide, including preferred sequences of the motif X1X2X3, are as set out above.

The polypeptide may comprise the sequence

QRKC,
FQRKC,
NFQRKC,
INFQRKC,
WINFQRKC,
KWINFQRKC,
RKCY,
RKCYY,
RKCYYF,
RKCYYFG,
RKCYYFGK,
RKCYYFGKG,
QRKCYY,
FQRKCYY,
FQRKCYYF,
FQRKCYYFG,
NFQRKCY,
INFQRKCY,
or
INFQRKCYY,

or the equivalent sequence from a non-human CD23 molecule, e.g. a mammalian CD23 molecule, such as a murine CD23 molecule.

It will be appreciated that the CD23 sequence may be produced as a fusion protein, e.g. to facilitate its detection in binding assays. For example, they may be tagged with an epitope for a known antibody, or fused to an enzyme or other protein capable of generating a signal. Preferably the remainder of the polypeptide or peptide does not comprise more than 5 contiguous amino acids from a CD23 sequence, and/or displays less than 20% sequence identity with a CD23 molecule as determined using the alignment algorithm and parameters detailed above.

The above-described preferred features of CD23-derived peptides apply particularly to peptides which are intended to interact with human αv integrins. However, not all of these peptides will necessarily interact with αv integrins from species other than humans. In particular, only a subset of these peptides is thought to interact with αv integrins from mice, rats, and other rodents.

Certain wild-type rodent CD23 sequences, including those of mouse and rat CD23, have Q at the position equivalent to R172 of human CD23. Those wild-type rodent proteins therefore carry the motif QKC instead of the RKC motif present in the human protein. When presented as part of a peptide, this motif can bind to, and induce signalling through, human αv integrins. However, it is believed that it does not bind appreciably to or signal through rodent αv integrins, particularly mouse αv integrins. A positive charge at the N-terminus of this tripeptide motif appears to be desirable for binding and signalling through rodent integrins.

Thus, where a peptide is intended for interaction with a rodent αv integrin, e.g. a murine or rat αv integrin, it is preferred that X3 is C, and X1 is a residue carrying at least a partial positive charge at physiological pH.

Thus X1 is preferably R or K, and most preferably R.

Preferably, X2 is also a residue carrying at least a partial positive charge at physiological pH. Thus, X2 is preferably R or K, and most preferably K.

When X2 is not a residue carrying at least a partial positive charge at physiological pH, it is preferably a neutral residue, i.e. a residue which does not carry any charge at physiological pH. Thus X2 may be Q, N, A, G, S, T, I, L, M, F, P, W, Y, V. In preferred embodiments, X2 is Q, N, A, G, S, T, V, L or I, and still more preferably A, Q, S or G.

Thus preferred sequences for a motif intended to interact with a rodent integrin include RKC, KKC and RRC.

Thus the peptides used for interaction with rodent integrins may be based on wild-type mouse or rat CD23, wherein the Q corresponding to R172 of human CD23 is substituted by a residue carrying at least a partial positive charge at physiological pH such as R or K, and preferably R.

When based on murine CD23, the peptide may comprise any of the sequences

QRKC,
FQRKC,
HFQRKC,
LHFQRKC,
WLHFQRKC,
NWLHFQRKC,
RKCY,
RKCYY,
RKCYYF,
RKCYYFG,
RKCYYFGK,
RKCYYFGKG,
QRKCYY,
FQRKCYY,
FQRKCYYF,
FQRKCYYFG,
HFQRKCY,
LHFQRKCY,
or
WLHFQRKCYY.

When based on rat CD23, the peptide may comprise any of the sequences

QRKC,
FQRKC,
HFQRKC,
LHFQRKC,
WLHFQRKC,
DWLHFQRKC,
RKCY,
RKCYY,
RKCYYF,
RKCYYFG,
RKCYYFGE,
RKCYYFGEG,
QRKCYY,
FQRKCYY,
FQRKCYYF,
FQRKCYYFG,
HFQRKCY,
LHFQRKCY,
or
WLHFQRKCYY.

The peptides and polypeptides of the invention are preferably capable of binding to αv integrins, particularly αvβ5, and are preferably capable of inhibiting binding between αv integrins (particularly αvβ5) and CD23. In some embodiments the peptides or polypeptides may be agonists of αv integrins, such as αvβ5.

The invention also provides a nucleic acid comprising an open reading frame encoding the peptides of the invention. The nucleic acids may be DNA or RNA, in single or double stranded form. The coding sequence may comprise naturally occurring CD23 sequence, e.g. part of the sequence of SEQ ID NO: 1, or may comprise wholly or partially artificial (i.e. non-naturally occurring) sequence.

Also provided are vectors comprising the nucleic acids of the invention in which the open reading frame is operably linked to regulatory sequences (e.g. promoter, enhancer and transcriptional terminator sequences, as well as translational control sequences) to allow transcription and translation of the peptide in a desired cell type. The skilled person will be able to design suitable vectors depending on the desired cell type, which may include bacterial, yeast, insect or mammalian cells. Also provided are host cells, including bacterial, yeast, insect and mammalian host cells, comprising such vectors.

The invention also provides the peptides and polypeptides described above, as well as nucleic acids encoding the same, for use in a method of medical treatment or diagnosis, and in particular for use in the treatment or diagnosis of cancer, and the treatment of inflammatory disorders.

The invention also provides a pharmaceutical composition comprising a peptide or nucleic acid as described above, in combination with a pharmaceutically acceptable excipient.

Pharmaceutical compositions may comprise, in addition to one of the active agents described above, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the peptides more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering the peptides directly, they could be produced in the target cells or in neighbouring cells by expression from an encoding gene introduced into the cells in a suitable vector as described above, e.g. in a viral vector. The vector could be targeted to the specific cells to be treated, or it could contain regulatory elements which are switched on more or less selectively by the target cells.

A composition may be administered alone or in combination with other treatments, either simultaneously or sequentially dependent upon the condition to be treated.

As explained above, it is believed that wild type rodent CD23 molecules (and particularly mouse CD23) do not bind with significant affinity to rodent αvβ5, although they may be able to bind to αvβ5 from other mammals such as humans. Similarly, rodent CD23 molecules do not appear to induce signalling via rodent αvβ5, but may be capable of causing signalling via human αvβ5. This may explain why murine CD23 has been reported not to possess many of the cytokine-like properties of human CD23.

As a consequence, rodents (or rodent cells) may not be ideal as animal models of human conditions which are thought to be mediated in whole or in part by CD23/αv integrin interactions.

Therefore, in a further aspect, the present invention provides a rodent cell which has been genetically modified so that it contains nucleic acid encoding a CD23 protein having R at a position corresponding to R172 of human CD23. Thus the cell is capable of expressing CD23 protein having R at a position corresponding to R172 of human CD23. This CD23 protein may be regarded as an “exogenous” protein as it is not naturally produced by the cell. Preferably the exogenous CD23 protein has the motif RKC at positions corresponding to positions 172 to 174 of human CD23.

The cells are preferably rat or mouse cells.

The exogenous CD23 protein may be a full-length membrane-bound CD23 protein, or a soluble form of CD23 protein lacking a transmembrane domain which will therefore be secreted from the cell.

The exogenous CD23 protein may be a CD23 protein from a different mammalian species, e.g. human CD23. Alternatively, it may be a modified form of the endogenous chromosomal CD23 gene having R at the position corresponding to R172 of human CD23. Other changes may also be introduced relative to the endogenous sequence. Preferably the exogenous protein has at least 80%, 85%, 90% or 95% identity to the murine or rat CD23 sequence given below.

```
Rat CD23 (GenBank accession nos. CAA51981.1 GI:313673):
    1 MEENEYSGYW EPPRRRCCCA RRGTQLVLVG LLTTVMVWLL ALLLLWHWET EKSLKQLGDA
  61 AIQNALQMSQ NLEELQAEQK QMKSQDSQLS QNLNELQEDL INVKSQNSEL SQNLNTLQED
121 LVNVKSQGLN EKRAASDSLE KLQEEVAKLW IEILMSKGTA CNVCPKDWLH FQQKCYYFGE
181 GSKQWIQAKF TCSDLEGRLV SIHSQKEQDF LMQHINKKES WIGLQDLNME GEFVWPDGSP
241 VGYSNWNPGE PNNGGQGEDC VMMRGSGQWN DAFCRSYLDA WVCEQLATCD LSAPLASVTP
301 TGPTPKNEP
Mouse CD23 (Isoform A; GenBank accession nos. AAB28791.2 GI:13236929):
    1 MEENEYSGYW EPPRKRCCCA RRGTQLMLVG LLSTAMWAGL LALLLLWHWE TEKNLKQLGD
  61 TAIQNVSHVT KDLQKFQSNQ LAQKSQVVQM SQNLQELQAE QKQMKAQDSR LSQNLTGLQE
121 DLRNAQSQNS KLSQNLNRLQ DDLVNIKSLG LNEKRTASDS LEKLQEEVAK LWIEILISKG
181 TACNICPKNW LHFQQKCYYF GKGSKQWIQA RFACSDLQGR LVSIHSQKEQ DFLMQHINKK
241 DSWIGLQDLN MEGEFVWSDG SPVGYSNWNP GEPNNGGQGE DCVMMRGSGQ WNDAFCRSYL
301 DAWVCEQLAT CEISAPLASV TPTRPTP
```

In the above mouse and rat CD23 sequences, the region corresponding to amino acids 155 to 191 of human CD23 is underlined, and the QKC motif corresponding to RKC 172-174 of the human sequence is shown in bold type. The extracellular domains of these proteins are believed to begin at H47 (rat) and H48 (mouse).

Preferably the exogenous CD23 protein comprises an RKC motif corresponding to positions 172 to 174 of human CD23.

In preferred embodiments the exogenous CD23 protein is identical to the endogenous protein apart from the residue corresponding to R172 of human CD23.

The exogenous CD23 protein is preferably capable of binding to rodent (e.g. mouse or rat) αvβ5 protein, and preferably also to human αvβ5.

The nucleic acid encoding the said CD23 protein may be extrachromosomal, e.g. on a self-replicating episomal expression vector, such as a plasmid. Alternatively it may be stably integrated into a chromosome of the cell.

The wild type cell has endogenous (chromosomal) CD23 genes encoding CD23 protein having a residue other than R at the position corresponding to R172 of human CD23. Typically these proteins have Q at that position.

The endogenous CD23 genes may remain intact, such that the cell expresses both exogenous CD23 (which has R at a position corresponding to R172 of human CD23) and endogenous CD23 (which does not).

Alternatively, the cell may not express endogenous CD23, and instead express only exogenous CD23. For example, the two endogenous chromosomal CD23 genes may be modified so that they do not express full-length endogenous CD23 protein. In other words, the cell is a “knock-out” for endogenous CD23. The coding sequence for the exogenous CD23 protein may be inserted into a chromosome at a different locus, or it may be introduced into the endogenous CD23 genes. Alternatively, the endogenous CD23 genes may be modified, e.g. by homologous recombination, so that the sequence encoding the residue corresponding to position 172 of human CD23 is replaced by a sequence encoding R at that position; i.e. the endogenous chromosomal genes are modified to express an exogenous CD23 protein. Further changes to the endogenous coding sequence may be made if required.

The invention further provides a transgenic rodent, comprising cells containing nucleic acid encoding an exogenous CD23 protein having R at a position corresponding to R172 of human CD23, as described above.

In preferred embodiments, the exogenous CD23 protein is the only CD23 protein expressed by the rodent. Thus both endogenous CD23 genes in the genome are preferably inactivated so that they do not produce full-length endogenous CD23 protein, or are modified to encode the desired exogenous protein. Mice in which the endogenous CD23 genes have been inactivated by knock-out techniques have been described by Stief et al. (J. Immunol. 152:3378 (1994)) and Yu et al. (Nature 369:753 (1994)).

Alternatively, the rodent may express CD23 from one or both endogenous CD23 genes in addition to the exogenous CD23 protein.

Methods for generating suitable transgenic rodents are well known to the skilled person.

The CD23 transgene, i.e. the nucleic acid encoding the exogenous CD23 protein, may be introduced into any suitable rodent genetic background. Many inbred rodent strains are used as models for particular human conditions. Transgenic versions of these rodents may therefore be used to mimic the effects of the αvβ5/CD23 interaction in the human disease.

Thus, by way of example, the CD23 transgene may be introduced into the NZB/W F1 mouse or the Bcl-2 transgenic mouse which are both models for systemic lupus erythematosus (SLE), the DBA1 mouse strain used in the collagen-induced arthritis model of rheumatoid arthritis, or the ApoE knockout mouse or LDL receptor knockout mouse, both of which are models for atherosclerosis.

These transgenic rodents may therefore be used to investigate the effects of candidate drug molecules, e.g. on development or progression of disease. The candidate drug molecules may have been identified as inhibitors of interaction between αvβ5 and CD23 by methods described elsewhere in this specification.

Thus the invention further provides a method of testing a substance for a prophylactic or therapeutic effect on an inflammatory disorder, comprising administering said test substance to a transgenic rodent as described above, said transgenic rodent being affected by, or suspected of being likely to develop, said inflammatory disorder.

The transgenic rodent may have a genetic background suitable for use as a model for rheumatoid arthritis, Sjogren's Syndrome, systemic lupus erythaematosus (SLE), sarcoidosis, endometriosis, thyroiditis or atherosclerosis

The invention further provides a method of testing a substance for a prophylactic or therapeutic effect on a neoplastic disorder, comprising administering said test substance to a transgenic rodent as described above, said transgenic rodent being affected by, or suspected of being likely to develop, said neoplastic disorder.

The neoplastic disorder may be a cancer which receives proliferative signals from an interaction between CD23 and αvβ5. For example, the method may use a transgenic mouse which has a pre-B cell cancer such as ALL, or a myeloma. An example of a mouse with a pre-B cell cancer would be a mouse inoculated with BA/F03 cells (see Palacios R and Steinmetz M, (1985) Cell 41:727-734).

The invention further provides a mutant of a wild type CD23 protein, wherein the wild type protein has a Q at a position corresponding to R172 of human CD23, the mutant comprising a residue having at least a partial positive charge at physiological pH at the position corresponding to R172 of human CD23. The mutant CD23 protein is capable of binding to rodent (e.g mouse or rat) αvβ5, and may also be capable of binding to αvβ3, 6 or 8.

The residue in the mutant at the position corresponding to R172 of human CD23 is preferably R or K, but is most preferably R.

Also provided is an isolated extracellular domain of the mutant protein described above. Also provided is an isolated peptide comprising at least 15 amino acids from said mutant protein, as long as the peptide comprises the motif RKC at the positions corresponding to 172 to 174 of human CD23. The peptide may comprise at least 20, 25, 30 or more amino acids from the mutant protein.

Preferably the mutant protein, extracellular domain or peptide has at least 90% sequence, and preferably at least 95% identity to the corresponding portion of the wild type rat or mouse CD23 sequence given above. In particularly preferred embodiments, the sequence of the mutant protein, extracellular domain, or peptide is identical to the wild type rat or mouse sequence except at the position corresponding to R172 of human CD23.

The mutant protein, extracellular domain or peptide may comprise additional sequences such as a signal peptide, transmembrane domain or intracellular sequence. These sequences may be wild type CD23 sequences or may be heterologous to CD23. For example, they may be derived from other proteins or may be synthetic. The protein, extracellular domain or peptide may be fused to additional non-CD23 oligo- or polypeptide sequences, such as epitope tags for recognition by antibodies, or purification tags to allow purification from a mixture of proteins.

The invention also provides an isolated nucleic acid comprising an open reading frame encoding a mutant protein, extracellular domain or peptide as described above. Also provided is a vector comprising said nucleic acid and a host cell comprising said vector.

### DESCRIPTION OF THE DRAWINGS

FIG. 1. The αvβ5 Integrin is a CD23 Receptor in SMS-SB Cells

FIG. 1A:—Low cell density (LCD) cultures of SMS-SB in PFHM were established with the indicated concentrations of recombinant 25 kDa sCD23 (panel i) or other cytokines and cellular proliferation (panel i) or ratio of viable to apoptotic cells (panel ii) determined. Normal cell density (NCD) cultures (5×10⁵ cells/ml) served as a control.

FIG. 1B:—Extracts of SMS-SB cells in OGP buffer, were passed over a BSA-Affigel column and the flow-through (F/T) collected and subsequently incubated on CD23-Affigel; aliquots of F/T and eluted fractions from both columns were electrophoresed under reducing conditions. Positions of molecular weight standard markers are noted. [³⁵S]-methionine-labelled proteins were visualised by fluorography (panel i), and unlabelled proteins were transferred to nitrocellulose and probed with anti-αv-specific (panel ii) or anti-β5-specific (panel iii) rabbit anti-peptide antibodies and binding visualised with protein A-HRP and ECL. Note that in FIG. 1Ciii, the antibody recognises a C-terminal peptide of αv and so detects a 25 kDa protein under reducing conditions.

FIG. 1C:—SMS-SB cells were stained (solid lines) with MAbs specific for αvβ5 (panel i), CD47 (panel ii), or αvβ3 (panel iii) and staining visualised with the appropriate secondary reagent (shaded area).

FIG. 2. Ligand-Selective Responses Regulated by αvβ5

Low cell density (LCD) cultures (2500 cells/well) of SMS-SB (Panel A) or Nalm-6 cells (Panel B) were propagated with the indicated concentrations of AMF anti-αv MAb (black bar) and IgG1 isotype control (grey bar); proliferation was measured by [³H]-thymidine incorporation. In Panel B, AMF 7 and IgG1 were used at 5 μg/ml. Panel C shows the effect of conformation-dependent anti-αvβ5 MAbs P1F6 and 15F11 anti-αvβ5 MAbs with corresponding IgG1 and IgG2a isotype controls on SMS-SB cellular proliferation. Panel D illustrates the effect of culture with BSA (white bar) Vn (black bar) or Fn (grey bar). All experiments were performed a minimum of three times.

FIG. 3. αvβ5 Binds a Specific Linear Sequence in CD23

FIG. 3A:—ELISA plates were coated with αvβ5 integrin, washed and blocked. Increasing amounts of recombinant sCD23 were added to wells and specific binding detected using anti-CD23 MAb followed by detection with HRP anti mouse IgG and tetramethylbenzidine (TMB) as substrate (panel i). The relative binding of SMS-SB cells to CD23- or BSA-Sepharose beads in the presence or absence of different anti-CD23 (BU38, M-L233) or anti-αvβ5 MAbs (P1F6, AMF7) was determined (Panel ii).

FIG. 3B:—A series of 83 synthetic biotinylated tridecapeptides, of the form biotinyl-SGSG-X₉, where the nonapeptide sequence was based on the 25 kDa CD23 sequence (residues 150-321) was captured on streptavidin-coated ELISA plates, and then probed with either purified αvβ5 integrin. Binding was assessed by addition of appropriate primary antibody and HRP-conjugated secondary Ab and TMB (Panel i).

FIG. 3C:—Binding of biotinylated peptides to pre-B cell lines was detected by addition of PE-streptavidin and flow cytometry; mean fluorescence index (MFI) data were derived using CellQuest software. The flow cytometric plots for binding of peptides #9 (black line), #11 (grey line) and #57 (shaded area) are illustrated (Panel i), and MFI data for binding of the noted peptides to SMS-SB (Panel ii), Blin-1 (Panel iii) and Nalm-6 (Panel iv) are shown. The unique nine amino acid sequence of each biotinylated tridecapeptide is shown with the RKC motif shared by αvβ5 binding peptides shown in bold (Panel ii).

FIG. 4. CD23-Derived Peptides Containing the RKC Motif are Biologically Active.

FIG. 4A:—LCD cultures of SMS-SB cells (2500 cells/well) were established in the presence of the indicated concentrations of peptides #9-#12 (black bars) and inverted sequence variants of peptides #9 and #11 (grey bars). Thymidine uptake was scored 72 hr after cultures were established. (* Note that the ‘vehicle’ control is shown only for peptide #9; all other vehicle controls for different solvents also gave incorporation values in the range of 3000-5000 cpm, which is comparable to untreated controls (white bars)).

FIG. 4B:—LCD cultures of SMS-SB cells were assembled in the presence of a 10-fold molar excess of either peptide #10, #11 or #57, before addition of either peptide #9 (Panel i), or peptide #12 or, as a negative control, peptide #57 (Panel ii); [³H]-TdR was assessed after 72 hr. In each case, the effect of stimulatory peptide alone is shown in the black bar, and the effect of excess individual competitor shown as grey bars; the effects of excess peptides alone on SMS-SB cells are shown in panel i (white bars).

FIG. 4C:—Binding of peptides to cells was assessed as described at FIG. 3B. Fluorescence histograms are shown for wild-type (shaded area), R172K (grey line) and R172Q (black line) mutants of peptide #11 (Panel i), and MFI data for wild-type, scrambled and single substitution mutants of peptides #9 (black bars) and #11 (grey bars) are shown in Panel ii).

FIG. 4D:—LCD cultures of SMS-SB cells (2500 cells/well) were established in the presence of the indicated concentrations of peptide #9 (black bars) or peptide #11 (grey bars) and variants where the arginine was converted to glutamine (R172Q) or lysine (R172K). Thymidine uptake was scored 72 hr after cultures were established.

FIG. 5. αvβ5 Integrin Binds the RKC Motif at a Site Distinct from the RGD-Binding Site.

FIG. 5A. Peptide binding experiments were established as in Panel 3C above. Panel i shows fluorescence histograms for binding of peptide in the absence (shaded area) and presence of a ten-fold excess of RGDS peptide; Panel ii shows MFI values for peptide binding determined in the absence (black bar) or presence (grey bar) of 10 μg/ml of RGDS. LCD cultures were established with no stimulus (white bar), with 10 μg/ml RGDS peptide (stippled bar), peptide #9 alone (black bar) or either peptide #9 or #12 together with 10 μg/ml RGDS (grey bars); [³H]-TdR uptake was assessed after 72 hr (Panel ii).

FIG. 5B. Binding and proliferation experiments were established as above. Binding of wild type peptide is shown as the shaded area in each panel, and the peptide variant indicated on the panel is displayed by the grey line; binding of irrelevant peptide (#58) is shown as a thin black line (Panel i). Binding data are presented as MFI values (Panel ii). LCD cultures of SMS-SB cells were stimulated with the indicated peptides at 10 μg/ml for 72 hr prior to addition of [³H]-TdR and determination of proliferation (Panel iii).

FIG. 6. αvβ5 Expression in Normal & Neoplastic Haematopoietic Cells

PBMC (Panel A) were stained simultaneously with biotinylated-anti-αvβ5 (visualised with SA-QR) and one of PE-anti-CD19, or FITC-anti-CD2, —CD4 or —CD8; insets show staining with lineage marker antibody and biotinylated isotype-matched control antibody plus SA-QR. The percentage value is for marker-positive cells also scoring positive for αvβ5. Normal human bone marrow (Panel B) was stained with PE-anti-CD19 and either control antibody (panel i) or biotinyl-anti-αvβ5 (Panel ii). PBMC from two ALL patients are shown in Panel C (i and ii). Panels D and E illustrate single-colour staining histograms for single representative ALL and B-CLL patients, respectively, for the indicated CD23 receptors. Panel F displays a scattergram showing the percentage of tumour cells positive for αvβ5 in three types of ALL (common-B cell ALL ‘cALL’, CD10⁺; T cell ALL ‘T-ALL’, and for CD10⁻ ‘null’-ALL ‘n-ALL’), and for B-CLL and AML cells.

### DETAILED DESCRIPTION OF THE INVENTION

### Methods

### Materials

Anti-CD47 (BRIC 126, IgG2b), anti-αvβ3 (LM609, IgG1), anti-αvβ5 (P1F6, IgG1; 15f11, IgG2a), and rabbit polyclonal anti-peptide antibodies specific for integrin αᵥ and β₅ subunits were from Chemicon, UK. Anti-αv/CD51 (AMF7, IgG1) was obtained from Beckman Coulter, High Wycombe, UK.

Radiochemicals and materials for enhanced chemiluminescence (ECL) were obtained from Amersham International plc, Amersham, England, and fine chemicals, including streptavidin-Quantum Red (SA-QR), horseradish peroxidase (HRP)-coupled protein A, cyanogen bromide-activate Sepharose beads, and octyl-β-D-glucopyranoside (OGP), were supplied by Sigma, Poole, England.

Normal peripheral blood mononuclear cells (PBMC) were obtained from volunteers, and B-CLL leukaemic samples from patients attending the haematology clinic, Western Infirmary, Glasgow, with appropriate ethical permissions. Archival ALL and AML diagnostic samples (blood or bone marrow), collected with ethical permission in connection with earlier MRC clinical trials, were drawn from the LRF Centre Leukaemia Bank (Institute of Cancer Research, London). The SMS-SB cell line was derived from a female patient presenting with ALL 39, and the Nalm-6 and Blin-1 cell lines were from laboratory stocks. Human bone marrow stromal cells were immortalised by retroviral transduction of the human telomerase (hTERT) gene⁴⁰.

### Cell Culture

Cell lines were maintained in RPMI-1640 medium supplemented with 10% (v/v) heat-inactivated foetal calf serum (FCS), 2 mM fresh glutamine, and penicillin and streptomycin, at 37° C. in a 5% CO₂ in air in a humidified atmosphere. Cytokines, obtained from R&D Laboratories, were used at 5-10 ng/ml, but had no effect over a wide dose-response range. Recombinant 25 kDa sCD23, encompassing residues Met¹⁵¹-Ser²³¹ with an N-terminal his₆-tag, was expressed in E. coli and affinity-purified by nickel chelate chromatography. SMS-SB cells were also propagated in protein-free hybridoma medium-II (PFHM, GIBCO-BRL, Paisley, Scotland), at >10⁵ cells/ml (“normal cell density”—NCD). Telomerised stromal cells were cultured in DMEM supplemented with 10% FCS and 2 mM fresh glutamine, and were sub-cultured once per week. In the experiments described, the passage number for the stromal cells was between 80 and 95, and cells were allowed to adhere for 24 hr prior to addition of lymphoid cells in co-culture.

For stimulation experiments, SMS-SB cells were cultured at 2500 cells/100 μl culture (low cell density, LCD) a seeding density at which the cells are prone to apoptose¹⁵. NALM-6 cells were washed extensively in PFHM prior to culture at 2, 500 cells/100 μl culture. Cultures were propagated, in the presence or absence of cytokines, MAbs or peptides, at 37° C. for 72 hours followed by addition of 0.3 μCi/well tritiated thymidine ([³H]-TdR) for 18 hours prior to harvest; incorporation was determined by liquid scintillation spectrometry. Apoptosis was determined by staining harvested cells with propidium iodide (PI) and Hoechst 33342 for 1 minute prior to analysis on a Coulter Elite flow cytometer as previously described¹⁵. For stromal cell/SMS-SB co-cultures, 6000 stromal cells were seeded into individual wells of a 96-well tray and allowed to adhere for 24 hr; anti-CD23 MAb or isotype matched control and SMS-SB cells were added to the wells to give a total culture volume of 100 μl. SMS-SB cells were added at 5, 25 or 50 cells per well and cultures were allowed to expand over a 2-4 week period.

BA/F03 cells were routinely cultured in RPMI-1640 medium supplemented with 10% (v/v) FCS, 1 mM fresh glutamine, antibiotics (penicillin and streptomycin) and 50 μM 2-mercaptoethanol (‘complete medium’; CM). For stimulation experiments, cells were either washed in this medium and used or were extensively washed in protein-free hybridoma medium prior to use in short-term assays. For the assays, cells were cultured at 2500-3000 cells/100 μl culture (low cell density, LCD) and propagated in the presence or absence of cytokines, MAbs or peptides, at 37° C. for 24-72 hours followed by addition of 0.3 μCi/well tritiated thymidine ([³H]-TdR) for 18 hours prior to harvest; incorporation was determined by liquid scintillation spectrometry.

In CM, we (like many others) found that a sub-nanomolar dose of IL-3 was sufficient to sustain BA/F03 growth (EC₅₀˜0.5 nM). IL-3 is believed to activate the Akt pathway leading to phosphorylation and inactivation of the pro-apoptotic Bad protein.

### Lymphocyte Isolation & Immunophenotyping

5×10⁵ cells, or 20-50 μl whole blood (pre-treated with erythrocyte lysis buffer: −0.17 M ammonium chloride, 10 mM potassium bicarbonate, 0.1 mM EDTA), were stained with either fluorescein-(FITC)-conjugated or unlabelled primary MAb for 30-60 minutes; unlabelled primary antibody was visualised using a secondary FITC-conjugated anti-mouse IgG or, in the case of biotinylated anti-αvβ5, using SA-QR. Phycoerythrin-(PE)-labelled anti-CD19 was used to identify B cells. Cells were analysed on a FACScan flow cytometer, collecting 10,000 events per sample and the data analysed using CellQuest software.

### Labelling and Affinity Isolation of Cellular Proteins

10⁷ SMS-SB cells were harvested, washed twice with serum-free RPMI 1640 medium, suspended in 0.5 ml of labelling medium (DMEM lacking cold methionine and supplemented with 100 μCi of [³⁵S]-methionine) and incubated at 37° C. for 3 hours. Five millilitres of labelling medium supplemented with 10% (v/v) FCS were added and the culture incubated overnight at 37° C. The cells were washed twice in ice-cold PBS, suspended in 1.5 ml ice-cold OGP extraction buffer (1% (w/v) OGP in 50 mM HEPES/KOH pH 7.4, 5 mM CaCl₂, 140 mM NaCl, 1 mM PMSF, 1 mM aprotonin, 1 mM leupeptin) and lysed with 40 strokes of a chilled glass homogeniser. The homogenate was centrifuged at 1000×g for 10 minutes at 4° C., and the resulting supernatant further centrifuged at 35, 000×g for 45 minutes at 4° C. In experiments employing unlabelled cells, extracts were prepared from 10⁸ cells in OGP buffer.

Cellular extracts were added to BSA-Affigel pre-equilibrated with OGP extraction buffer and incubated at 4° C. for 6 hours. The matrix was pelleted, unbound proteins recovered and added to pre-equilibrated sCD23-Affigel and incubated at 4° C. overnight. The sCD23-Affigel was pelleted and the unbound fraction retained. Both matrices were exhaustively washed, specifically bound material eluted by boiling in sample buffer and subjected to SDS-PAGE under reducing conditions on a 10% (w/v) acrylamide gel. Radiolabelled proteins were visualised by fluorography⁵⁸. Unlabelled protein eluates were transferred to nitrocellulose membranes and probed with anti-VnR component antibodies, followed by an HRP-labelled secondary antibody and ECL.

### Binding of SMS-SB Cells to CD23-Coupled Sepharose Beads

10⁵ SMS-SB cells were added to 15 μl CD23- or BSA-coupled Sepharose beads and mixed gently for 30 minutes in the presence or absence of 0.5 μg anti-CD23 or anti-integrin MAb, or isotype control antibody, and the number of cells associated with each bead in 10 fields per slide was determined under the light microscope. Data are shown as fold-increase in cell binding to CD23-sepharose compared to BSA-sepharose, normalised to binding reactions in the absence of any immunoglobulin.

### Peptide Biochemistry

A library of 83 overlapping nonapeptides encompassing residues 151-321 of the 25 kDa sCD23 sequence was custom synthesised by Mimotopes Inc (Chester, UK); mutant peptides were synthesised by the same firm. Each peptide was synthesised as tridecapeptide comprising a unique CD23-derived nonapeptide sequence, plus a common N-terminal tetrapeptide extension (SGSG) to which a biotin moiety was attached. Each unique nonapeptide sequence had a two-residue C-terminal offset relative to its immediate neighbour. Aliquots of biotinylated peptides were captured on individual wells of a 96-well streptavidin-coated ELISA tray (Mimotopes, Chester, UK); binding of integrin was determined by addition of 0.2 μg of purified αvβ5 integrin to each well followed by addition of the P1F6 MAb and HRP-anti-Mouse IgG to quantitate binding. For Ab binding, aliquots of Ab were added and binding scored by addition of appropriate HRP-conjugated secondary Ab.

Binding of peptides to cells was visualised by treating cells exposed to biotinylated peptides with fluorochrome-conjugated streptavidin and scoring binding by flow cytometry; mean fluorescence intensity data were obtained using the CellQuest programme. In culture experiments, peptides were used in the 10 nM to 100 mM concentration range and appropriate solvent vehicle controls (e.g., acetonitrile, DMSO) were always performed.

### Flow Cytometry

5×10⁵ human U937 or murine RAW 264.7 monocytic cells were treated with biotinylated peptide at concentrations between 1 ng/ml and 1 μg/ml. Most experiments used 0.2 μg/ml. Peptide was added to 100 μl of cells for 30-60 minutes on ice, washed with phosphate-buffered saline (PBS), then resuspended in 300 μl PBS and exposed to 1 μg strepatavidin-phycoerythrin (or PE-Cy5-streptavidin in some experiments). After a further incubation on ice (30-60 minutes), the cells were washed with PBS and then analysed on a FACScan flow cytometer, collecting 10,000 events per sample. Data was analysed using CellQuest software.

For competition experiments, non-biotinylated peptide was present at approximately a 10-fold molar excess relative to the biotinylated probe peptide. Note that the probe peptides are of the form biotin-SGSG-X₉ (where X₉ is the unique nonapeptide sequence based on CD23 and SGSG is a tetrapeptide linker), while the competitors are of the form X₉ (i.e., contain only the CD23-derived sequence).

### Western Blotting Using Peptide Probes

Approximately 0.5 μg of purified integrin protein (αvβ3, αvβ5 and α5β1, all purified from human placenta and purchased from Chemicon) was electrophoresed on 10% (w/v) acrylamide gels using the Laemlli discontinuous buffer system. The stacking and separating gel and sample buffer mixes contained SDS, but lacked any reducing agent (i.e., had no dithiothreitol or 2-mercaptoethanol). This configuration ensures that the two component chains of the non-covalent heterodimeric integrin complexes are separated (e.g., to αv and P5), but that any intrachain disulphide bonds are not hydrolysed. Thus, in this system, the αv chain will migrate at ˜150 kDa since the 125 kDa and 25 kDa elements of the mature αv chain derived from the single large precursor protein will remain disulphide-bonded.

After electrophoresis, the proteins were transferred to nitrocellulose filters and, after blocking (with 10% milk powder in appropriate buffer), the filters were treated for 2-3 hours with 50 μg of probe peptide (e.g., biotinylated peptide #9). The filters were washed with 4-5 changes of buffer over a 2-3 hour period on a rocking platform, then treated with HRP-streptavidin for 60 minutes and again washed 4-5 times. Binding was visualised using the SuperSignal West enhanced chemiluminescence system.

For competition experiments, non-biotinylated peptides were included at a two- to five fold molar excess at the step where the probe peptide was added to the filters. The filters were pre-incubated with unlabelled peptide for one hour prior to exposure to biotinylated peptide; the latter was added to the buffer already containing the unlabelled peptide so that the non-biotinylated peptide was continuously present.

### Results

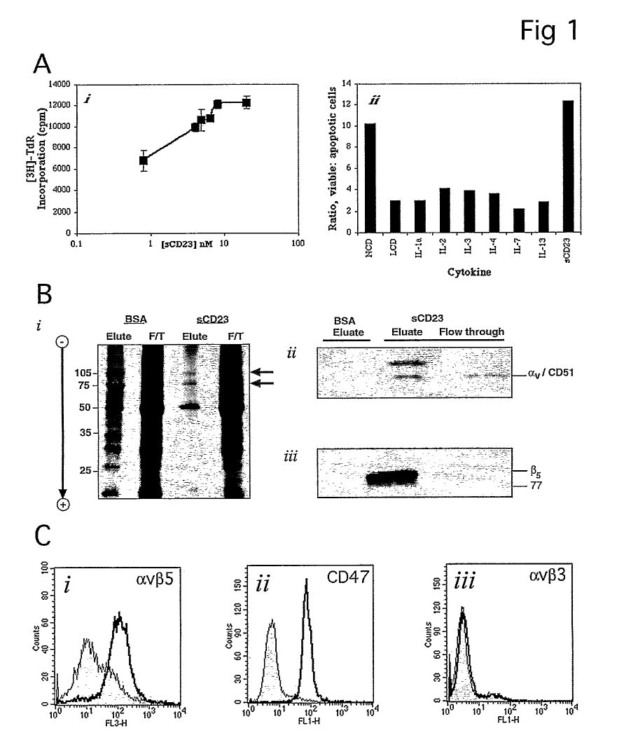

We have previously shown¹⁵ that recombinant soluble CD23 (sCD23) sustains growth and blocks apoptosis in a dose-dependent manner in low cell density (LCD) cultures of a human pre-B cell-like cell line, SMS-SB 39, derived from a female acute lymphoblastic leukaemia (ALL) patient (FIG. 1A). SMS-SB cells do not express CD21, the P2 integrins CD11b-CD18 or CD11c-CD18¹⁵, or the VnR αvβ3 (FIG. 1Ciii). In order to identify the CD23 binding structure, lysates of [³⁵S]-methionine-labelled SMS-SB cells were passed over a sCD23-Affigel column and bands of Mr ˜120 kDa and ˜80 kDa, consistent with those of mature αᵥ and β₅ integrin chains, respectively, are specifically enriched in the eluates (FIG. 1Bi). Western blots of SMS-SB cellular proteins specifically eluted from sCD23-Affigel matrices probed with anti-αᵥ and anti-β₅ antibodies (FIGS. 1Bii and 1Biii, respectively), confirm that both αᵥ and β₅ species bound to the matrix. Note that the polyclonal anti-αv antibody binds to a C-terminal epitope on αv that is located on the 25 kDa fragment generated during biosynthetic maturation of αv⁴¹. No β3 integrin protein is detected in whole cell extracts of SMS-SB cells or in eluates from sCD23 affinity columns (data not shown). SMS-SB cells stain with both the P1F6 MAb (FIG. 1Ci) that recognises the assembled αvβ5 heterodimer⁴², and anti-CD47 VnR-associated protein MAbs (FIG. 1Cii). The cells do not stain with the αvβ3-specific LM609 MAb (FIG. 1Ciii)^{42,43}. RT-PCR analysis of SMS-SB RNA yields amplicons of the appropriate sizes for CD47, CD51/αᵥ, β1 and β₅, but no correctly-sized PCR products are detected for β₃, β₆ or β₈ coding sequences (data not shown). The data demonstrate that CD23 binds to the αvβ5 integrin in SMS-SB cells and that this interaction regulates survival and growth of a model pre-B cell line.

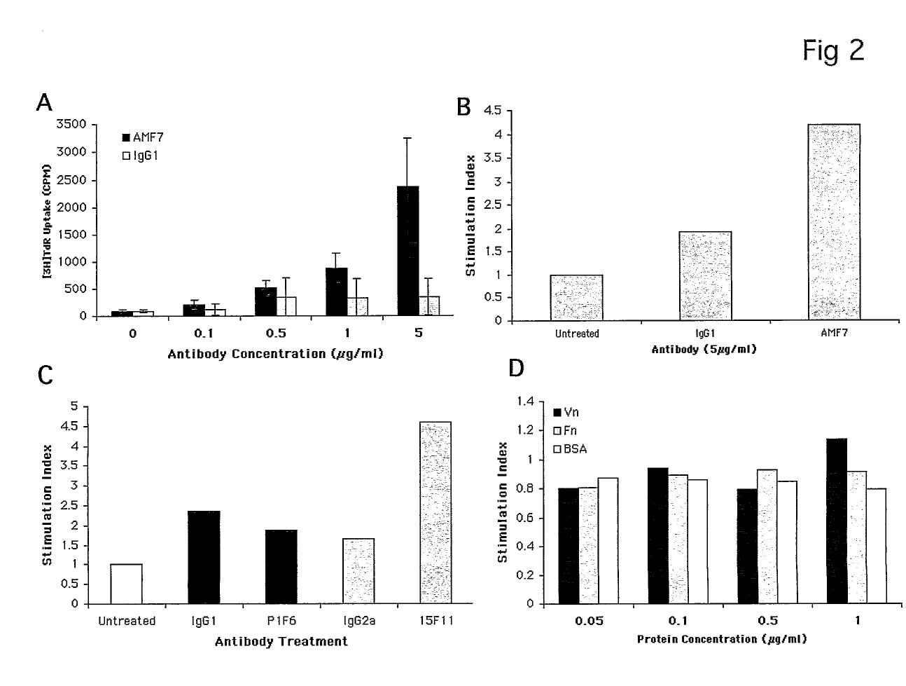

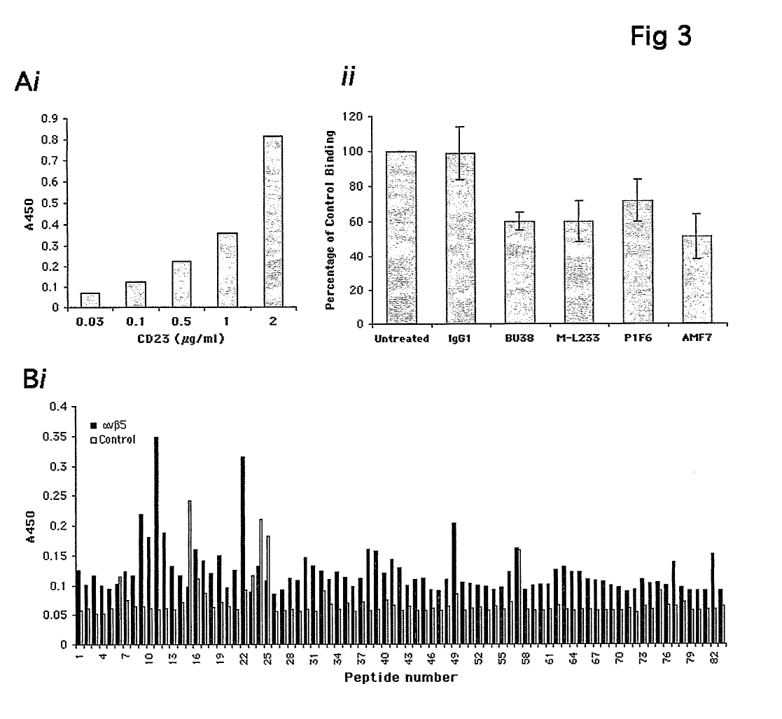

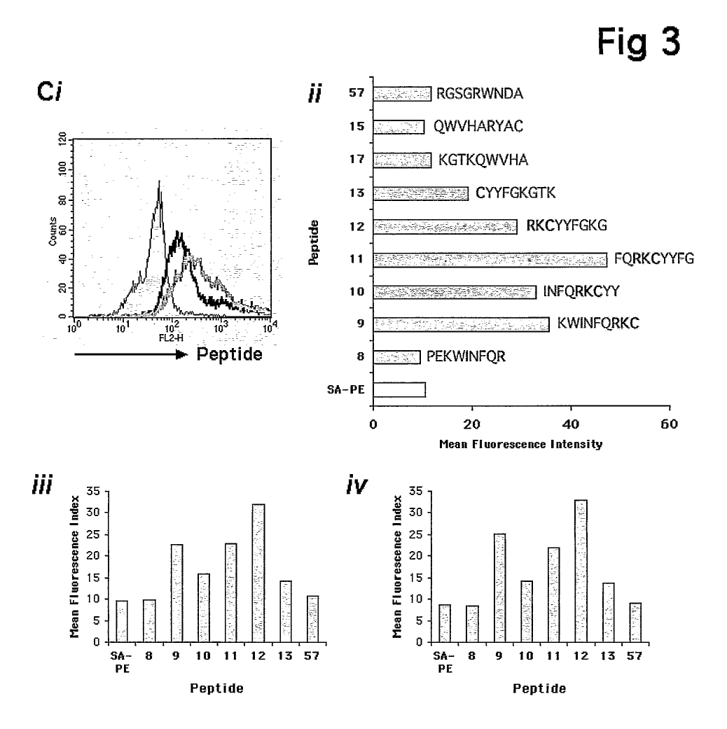

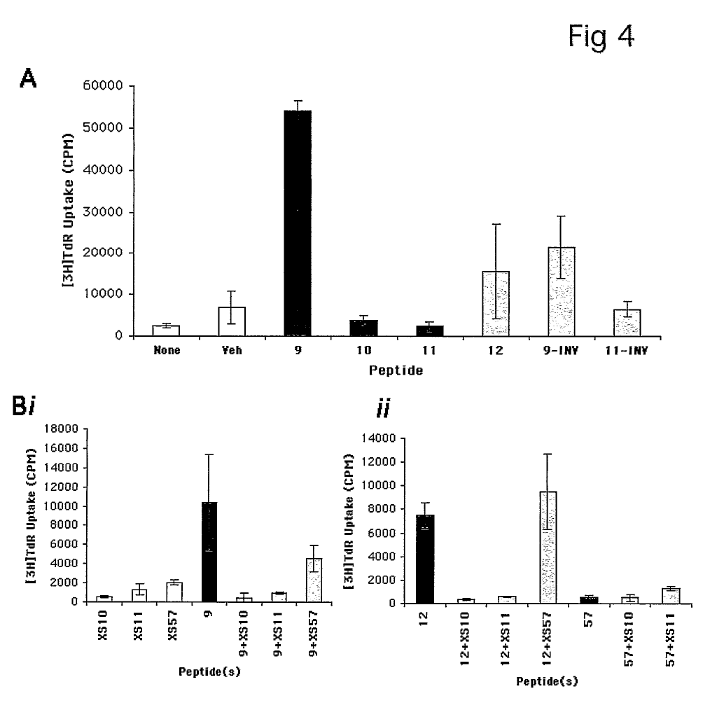

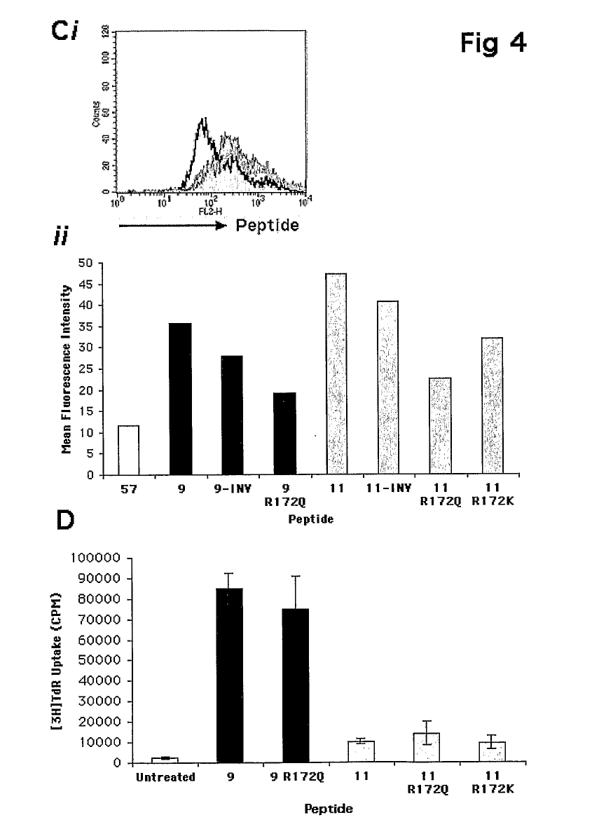

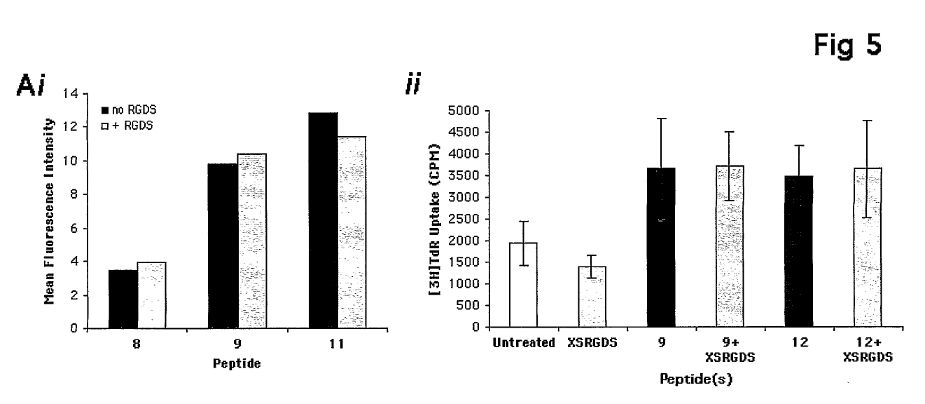

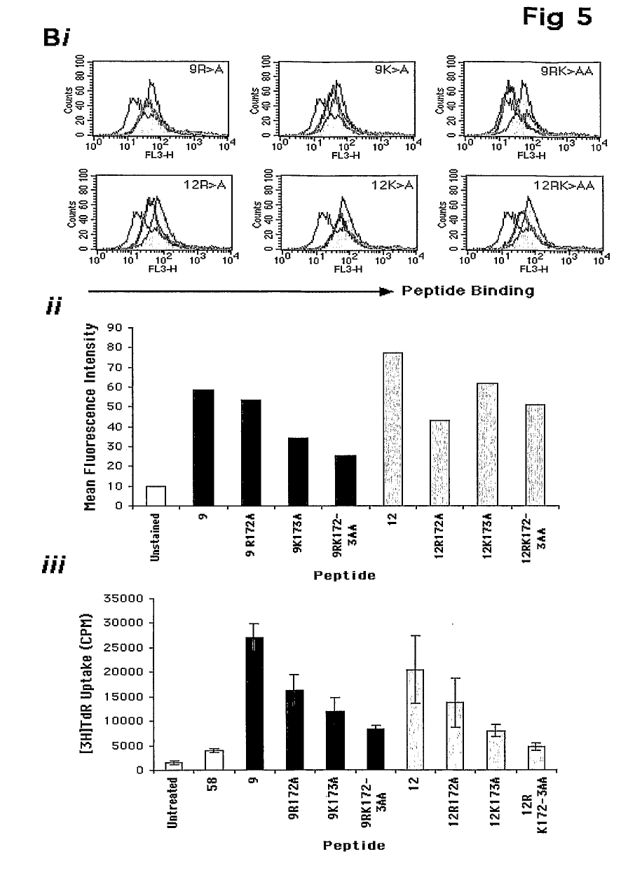

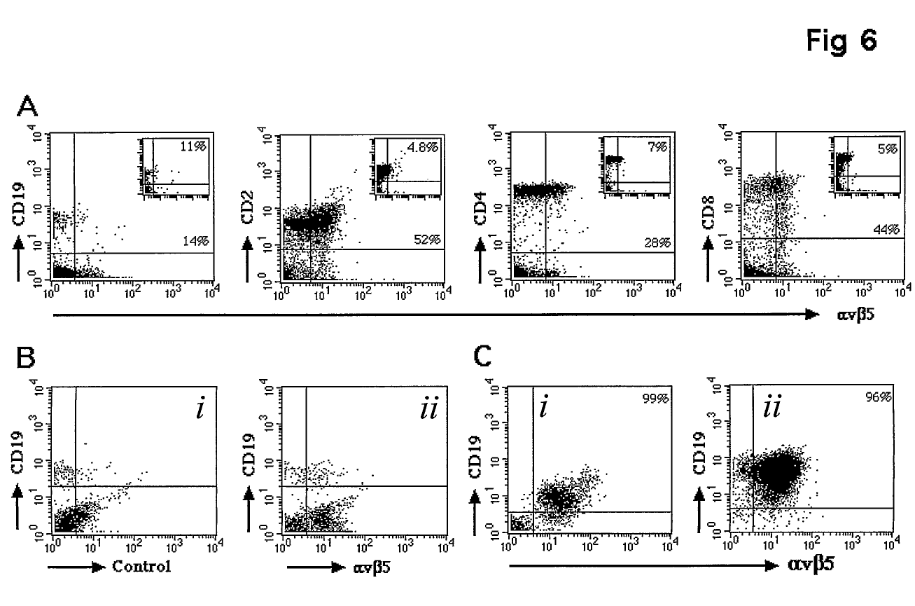

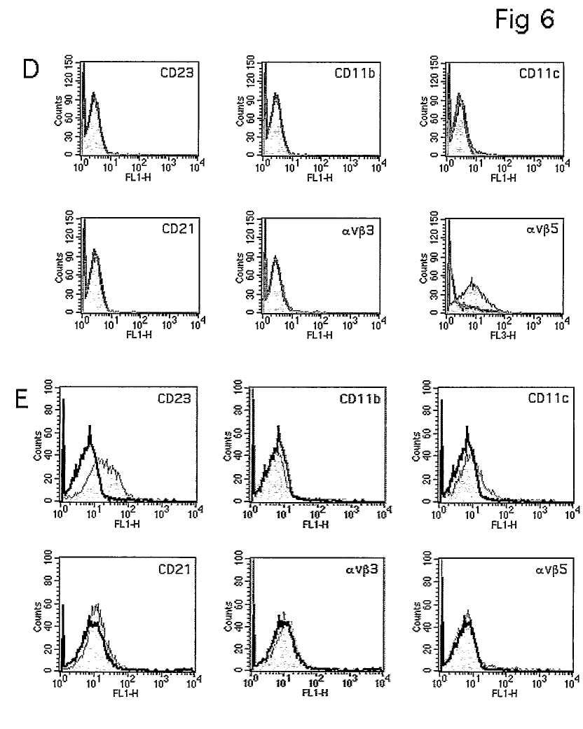

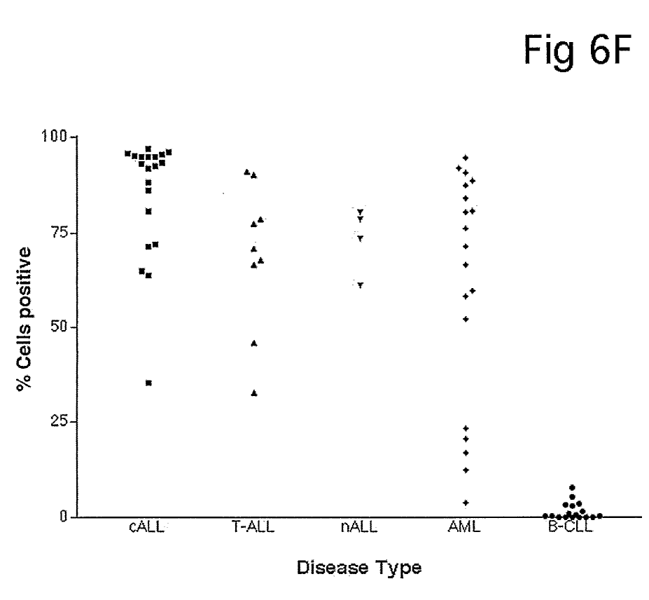

The data of FIG. 1 demonstrate that CD23 binds the αvβ5 VnR and suggest this integrin sustains cell growth. The anti-αv MAb AMF7 induces a strong dose-dependent increase in thymidine incorporation in both SMS-SB cells (FIG. 2A) and, importantly, in a second pre-B cell line, NALM-6 (FIG. 2B). We next used as stimulants the P1F6 and 15F11 MAbs that recognise distinct epitopes dependent upon complete assembly of the αvβ5 heterodimer. The 15F11 MAb, whose binding to αvβ5 is insensitive to ligand⁴⁴, sustains SMS-SB cell survival; however, the P1F6 reagent (that blocks binding to Vn) fails to enhance growth of SMS-SB cells (FIG. 2C). The data confirm that the αvβ5 integrin regulates cell survival in pre-B cells. The observation that the 15F11 and P1F6 MAbs also elicit different responses in SMS-SB cells (FIG. 2C) suggests the pro-survival effect is ligand-selective, an interpretation supported by the fact that neither Vn nor Fn sustain the growth of SMS-SB cells (FIG. 2D). The finding that neither Vn nor Fn stimulate pro-survival responses suggests that CD23 interacts with αvβ5 at a site distinct from that used by the integrin to bind RGD-containing matrix proteins.

ELISA-type assays demonstrate dose-dependent binding of recombinant 25 kDa sCD23 to immobilised αvβ5 (FIG. 3Ai) and vice versa (data not shown), and binding of SMS-SB cells to agarose beads coated with 25 kDa sCD23 is inhibited by MAbs directed against either αvβ5 or CD23 (FIG. 3A ii). We next used purified αvβ5 protein to probe a library of 83 biotinylated peptides containing overlapping nonapeptide sequences based on the 25 kDa sCD23 sequence. Purified recombinant αvβ5 protein binds specifically to a group of four peptides (peptides #9-#12) near the N-terminus of 25 kDa sCD23 and to a further peptide (#17) (FIG. 3Bi). In multiple experiments, the αvβ5 protein bound only to peptides #9-#12, with no other peptide showing a consistent binding to the integrin. The conformation-dependent M-L233 anti-CD23 MAb bound to none of the 83 peptides, but a goat polyclonal Ab directed against the C-terminus of CD23 binds strongly to peptide #82 (data not shown). Flow cytomety shows that peptides #9 and #11 bind strongly to SMS-SB cells but peptide #17, which gives a minor signal in the in vitro binding assay to integrin, does not bind to cells (FIG. 3Ci). The sequences of the peptides used are shown on FIG. 3Cii and the sole feature shared by all peptides with αvβ5 binding activity is a tripeptide motif of arg-lys-cys (RKC, embolded on the figure). Peptides #9-#12 also display strong binding to two other pre-B cell lines, Blin-1 (FIG. 3C iii) and NALM-6 (FIG. 3Civ). Four other CD23-derived peptides, chosen either randomly (#57), on the basis of being immediately adjacent to (#8 and #13), or having a similar charge (#15) to peptides with binding activity fail to bind any cell line (FIG. 3C ii). Peptides #61-#63, which contain an RKL sequence, or peptides #78-#80, which possess the ‘inverse RGD’ sequence, do not bind purified αvβ5 (FIG. 3B) or cells (data not shown). RKC is the minimum requirement for binding to the αvβ5 integrin.

In proliferation assays, peptides #9 and #12 stimulate thymidine incorporation by SMS-SB cells (FIG. 4A); peptides #10 and #11 have minimal effects. The data show that the peptides derived from the region of the CD23 protein recognised by αvβ5 integrin not only bind specifically to cells, but also, in some instances, mimic the effect of CD23 itself in promoting growth of SMS-SB cells. Inverting the sequence of peptide #9 (9-INV) reduces substantially, but does not ablate, its ability to elevate thymidine incorporation (FIG. 4A). The biological activity of sequence-inverted peptide #11 was minimally altered (FIG. 4A). Since peptides #10 and #11 bind to cells but do not stimulate thymidine incorporation, we next probed the potential antagonistic function of peptides #10 and #11 by pre-treating SMS-SB cells with one of these peptides before addition of either peptide #9 or #12, both of which drive SMS-SB cell growth. Each of peptide #10 and #11 reduces the growth stimulatory effect of either peptide #9 (FIG. 4Bi) or #12 (FIG. 4Bii) to background levels, but an irrelevant peptide, #57, is without significant antagonistic effect. Neither peptide #10 or #11 had any positive or negative effect on the ability of peptide #57 to influence SMS-SB cell growth (FIG. 4B ii). These data indicate that peptides #10 and #11 can antagonise specifically the growth-promoting activities of peptides #9 and #12.

The sequence encompassed by the RKC-containing peptides #9-#12 is equivalent to residues lys¹⁶⁶-gly¹⁸⁰ in the full length CD23 protein, with the RKC motif located at residues 172-174. Comparison of available CD23 sequences indicates that the presence of arginine at position 172 (arg¹⁷²) is unique to human CD23, suggesting that this residue was critical for binding. Inversion of the peptide sequence of peptides #9 and #11 (9-INV and 11-INV) does not reduce greatly binding of peptides to SMS-SB cells, although substitution of arg172 with gln (R172Q) in both peptides does impair binding (FIG. 4Ci and ii). Similar data are obtained for binding to Nalm-6 and Blin-1 cells (data not shown). Strikingly, the capacity of peptide #9 to promote thymidine incorporation in SMS-SB cells is not significantly reduced by the R172Q substitution (FIG. 4D). Substitution of arg in peptide #11 to either lys or gln had no effect on its inability to sustain SMS-SB cell growth. These data suggest that although binding of peptide #9 to cells is reduced by replacement of arg¹⁷² with gln, the mutant peptide retains the capacity to sustain the growth of SMS-SB cells.

The data of FIG. 4 show that arg172 is not required for peptide biological activity, suggesting that the αvβ5 integrin recognises the RKC motif in CD23 via binding site distinct from that used to capture RGD-containing ligands. Consistent with this hypothesis, an excess of a tetrapeptide (RGDS) containing the prototypic integrin binding tripeptide motif, RGD, neither impedes peptide binding to SMS-SB cells (FIG. 5Ai and ii), nor inhibits thymidine incorporation induced by peptides #9 and #12 (FIG. 5A ii). These data confirm that arg172 is dispensable for growth-sustaining activity of peptides #9 and #12 and that αvβ5 integrin does not recognise CD23 using the RGD-binding site. The minimal RKC sequence recognised by the αvβ5 integrin resides in a region that is basic in character; the human CD23 sequence is QRKC while the murine equivalent is QQKC (and the R172Q variant of peptide #9 both binds cells and stimulates thymidine incorporation). To test the hypothesis that αvβ5 integrin recognises a basic region in CD23, we substituted arg172 and lys173 in the agonist peptides #9 and #12 with alanine either singly or together. Single alanine substitutions reduce peptide binding to different extents (FIG. 5Bi and ii); the R172A variant of peptide #9 retains more binding ability than the K173A equivalent, but the reverse is true for variants of peptide #12. Double alanine substitutions reduce peptide binding to SMS-SB cells significantly for both peptide, but particularly so in peptide #9 (FIG. 5Bi and ii). In proliferation assays, the R172A and K173A variants of both peptides#9 and #12 promote SMS-SB cell growth less effectively than wild type peptides; the K173A variants are consistently less growth promoting than the R172A equivalents (FIG. 5B iii). The double alanine substitution variants do not promote growth to a significant level; proliferation levels are close to that driven by peptide #58 that does not bind to SMS-SB cells (FIG. 5B iii). These data are entirely consistent with the interpretation that αvβ5 recognises the basic character of the RKC motif using a binding site distinct from the RGD-binding site, and also help to explain ligand-selective signalling via the αvβ5 integrin in B cell precursors.

The αvβ5 staining pattern of lymphocytes derived from peripheral blood, bone marrow and two representative ALL patients show that although subsets of total, CD4⁺ and CD8⁺ peripheral T cells express αvβ5, there is essentially no αvβ5 expression on CD19⁺ B lymphocytes in normal individuals (FIG. 6A). Normal human bone marrow contains CD19⁺ cells that are αvβ5⁺ (FIG. 6B), and two ALL samples display substantial populations of αvβ5⁺ cells in peripheral blood (FIG. 6Ci and ii). The finding of high levels of αvβ5+/CD19⁺ B cells in the peripheral blood of ALL patients contrasts strikingly with the absence of such cells in the blood of normal subjects. Analysis of the expression patterns of CD23 receptors on cells from representative ALL and B-chronic lymphoblastic leukaemia (B-CLL) patients demonstrates that αvβ5 is the only CD23 receptor expressed on ALL cells; αvβ3 is not present (FIG. 6D). In contrast to ALL, no αvβ5 (or αvβ3) expression is detected in any B-CLL sample (FIG. 6E); the cytometric data for B-CLL are totally supported by analysis of CD23 receptor transcripts (data not shown). We next compared proportions of αvβ5⁺ cells in three distinct ALL types with cohorts of ˜20 B-CLL and acute myeloblastic leukaemia (AML) samples. The data demonstrate that αvβ5 is universally expressed on ALL-derived samples (regardless of the lineage of the tumour cells, or age of the patient) and is present on the majority of AML samples at variable levels; αvβ5 is consistently absent from B-CLL cells (FIG. 6F). The patterns of expression of αvβ5 in ALL and B-CLL reflects those found in non-malignant B cells (FIGS. 6A-C), with αvβ5 being found exclusively on precursor cell-derived leukaemias.

Competition experiments were performed to investigate whether binding of peptides containing an RKC motif to αv integrins can be specifically inhibited by similar peptides. Peptides #9-12 derived from 25 kDa sCD23 contain the RKC motif required for binding to the αv integrin family. Binding of biotinylated peptide #9 to pre-B cell lines (as exemplified by SMS-SB cells) can be inhibited by inclusion of non-biotinylated forms of peptide #10 in the assays; this tallies well with the ability of peptide #10 to inhibit the capacity of peptide #9 to stimulate SMS-SB cell growth and survival. Similar inhibition of binding of biotinylated (probe) peptide #9 by non-biotinylated variants is also observed in monocytic cell line models (e.g., the U937 cell line).

In vitro cell culture experiments show that inclusion of an RGDS tetrapeptide in cultures containing biotinylated peptide #9 fails to block binding of peptide #9 to SMS-SB cells or the ability of peptide #9 to promote cell growth. These data suggest the αv integrin uses a site distinct from the well-understood “RGD-binding site” to capture the basic RKC motif. This conclusion is supported by the observation that monoclonal antibodies that neutralise RGD-dependent adhesion do not mimic the ability of CD23 to sustain cell growth, while MAbs directed to sites not linked to adhesion do mimic CD23 activity in SMS-SB cells. Finally, the crystallographic model of αvβ3 structure illustrates unequivocally that an RKC motif could not be accommodated in the RGD binding site.

In order to investigate which part of the αv integrin molecule interacts with peptides containing an RKC motif, purified αvβ5, αvβ3 and α5β1 integrin proteins were electrophoresed under non-reducing conditions, transferred to nitrocellulose membranes and probed with biotinylated peptide #9 using HRP-streptavidin and enhanced chemiluminescence to visualise binding. Peptide binding to β1, β3 and β5 chains was readily detectable, but binding to the α5 or αv subunits was weak or absent. Binding of biotinylated peptide #9 to β3 and β5 chains was blocked by inclusion of excess non-biotinylated, RKC-containing peptides in the binding reactions.

These data suggest that RKC-containing peptides interact specifically with the β chains of the αv integrin family and not with the αv subunit. Moreover, since the RGD-binding site for recognition of adhesion ligands requires elements from both the αv and β(3) subunit to form the binding site, our western blotting data further demonstrate that the αv integrins have a second ligand binding site that is distinct from the RGD-binding site. The data also suggest that free β chains might be used in in vitro assays for ligands interacting with this second integrin binding site or for screening small molecules that might impede the interaction of our RKC-containing probe peptides with isolated β chains.

In vivo exploitation of compounds that block the prototype CD23-αvβ5 interaction will require a good animal model for validation. We used the IL-3-dependent murine pro-B cell line, BA/F03 cell line as a model equivalent to the human SMS-SB cell line. Peptides #9-#12 bind well to BA/F03 cells and peptide #9 can sustain BA/F03 cell survival in the absence of IL-3, but only at high doses and even then only rather weakly. However, if the cells are treated with a sub-optimal dose of IL-3, peptide #9 overtly stimulates growth. Murine CD23 does not possess an RKC motif at the position equivalent to that found in human CD23 but rather has a QKC (gln-lys-cys) sequence. A peptide #9 variant containing the murine sequence (“peptide #9 R172Q”) fails to bind BA/F03 cells and fails to have any growth-sustaining effect either by itself or in combination with IL-3. Peptide #11 R172Q also fails to bind to BA/F03 cells. The R172Q substitution also greatly reduces binding of peptide #9 to the RAW murine macrophage cell line.

Murine CD23 is believed to lack cytokine-like activity. These binding data indicate that the presence of a non-basic amino acid, glutamine, at the position equivalent to 172 of human CD23, may partly explain this lack of cytokine activity mediated via αv integrins. In vivo models for analysis of agents perturbing the CD23-αvβ5 interactions (and others like it) may therefore require cell lines expressing CD23 molecules having a positive charge at the position equivalent to R172 in human CD23. Murine cells transfected with CD23 containing a Q to R substitution, or with human CD23, may suffice. A transgenic line expressing human CD23 or murine CD23 having a Q to R substitution may enable human conditions in which the αvβ5-CD23 is implicated to be mimicked in an animal model in vivo, allowing better validation of potential drug molecules than is currently possible.

### Discussion

These data demonstrate that the αvβ5 integrin is a CD23 binding protein that regulates growth of pre-B cell lines. The observation that anti-αvβ5 MAbs that block adhesion to matrix proteins cannot mimic CD23 action, while MAbs directed to other αvβ5 epitopes can sustain cell growth, supports the interpretation that the αvβ5 integrin mediates distinct responses depending on the ligand encountered. The αvβ5 integrin recognises an RKC tripeptide motif that resides in a small basic region of the CD23 protein using a site that is distinct from the RGD-binding structure, and sensitive to basic character, rather than precise sequence. This explains why CD23, but not Vn or Fn, drive pro-survival responses in pre-B cell lines.

The data of FIG. 2 demonstrate that different αvβ5 ligands (CD23, Vn and Fn) and MAbs directed to distinct epitopes of αvβ5 itself elicit different characteristic responses in pre-B cell lines, potentially by acting via distinct binding sites on αvβ5. There are precedents for ligand-selective-signalling via αvβ3. In monocytes, CD23 promotes pro-inflammatory cytokine synthesis while Vn drives cell spreading but no cytokine production¹⁷. Similarly, in K562 cells, αvβ3 adheres with different affinities to Fn and Vn via processes regulated by, and resulting in activation of, distinct signalling pathways⁴⁵. In our system, the finding that RGD-containing peptides fail to inhibit either peptide #9 or #12 binding to cells or their ability to sustain cell growth (FIGS. 4 and 5) confirms that αvβ5 binds CD23 using a structure distinct from the RGD-binding site. The X-ray crystallographic model of αvβ3 integrin in association with an RGD-containing cyclic pentapeptide fully supports this interpretation. The RGD-peptide arg is secured by a bidentate salt link with asp²¹⁸ and a second such link with asp¹⁵⁰ from the αv chain³⁷, while the asp side chain is secured by contacts with Tyr¹²², arg²¹⁴ and asn²¹⁵ from the β3 chain, plus contact with a Mn²⁺ ion³⁷. The peptide gly residue resides at the interface between the αv and β3 domains making multiple hydrophobic interactions, including a dominant contact with the carbonyl oxygen of arg²¹⁶ of αv³⁷. The long side chain of the RKC peptide lysine would clash seriously with this carbonyl moiety and so preclude stable insertion of the RKC motif into the RGD-binding site.

Integrins recognise sequences other than RGD, and the αvβ5 integrin recognises the HIV Tat protein via a non-RGD motif that is basic in character³⁸ Recognition of basic domains has been established for other integrins including α2β1 integrin which binds a basic tetrapeptide sequence (RKKH) derived from the snake venom metalloprotease, jararhagin, a potent inhibitor of platelet binding to collagen⁴⁶. The data of FIGS. 4 and 5 illustrate that substitution of arg172 to gln (the latter being found in murine CD23) can reduce binding of peptide #9 to SMS-SB cells slightly but has no striking effect on stimulation of thymidine incorporation. Moreover, replacement of arg172 alone with ala only slightly reduces both peptide binding to cells and growth sustaining capacity; conversion of lys173 to ala gives a more marked reduction in binding and stimulation, but activity remains. It is only when both basic residues are substituted with non-polar alanine that both cell binding and growth sustaining ability is lost. These data argue strongly that the αvβ5 integrin recognises the basic nature of the RKC-containing region of CD23 and does so via a binding site distinct from that used to capture RGD-containing ligands. Recognition of the RKC-containing region is not sequence-specific, since peptides containing the QKC sequence based on murine CD23 bind and retain activity; moreover, the inverse sequence of peptide #9 (CKRQFNIWK) preserves basic character but not sequence and retains some residual binding and growth-stimulating properties. This is entirely in keeping with data from the binding of αvβ5 to the HIV Tat protein that showed neither poly-lysine or poly-arginine peptides bind αvβ5 affinity columns as well as the Tat or Vn basic domain peptides.

The RKC motif recognised by the αvβ5 integrin is located at positions 172-174 of the human CD23 protein. Models of the CD23 structure based on the mannose binding protein C-type lectin^{49,50} do not contain the full RKC motif, but suggest it is located just beneath the lectin head domain and at the upper reaches of the coiled-coil stalk region of the CD23 protein. The precise structure of this region remains to be resolved. The regions of CD23 needed for binding to IgE and CD21 reside in the lectin domain, and are known to overlap but be distinct¹⁰; trimeric sCD23 is capable of binding both CD21 and IgE simultaneously. The location of the RKC motif for αv integrin binding is, therefore, distinct again from the CD23 structures required for IgE and CD21 engagement. It is therefore possible that CD23 could interact simultaneously with three different ligands; IgE and CD21 will bind at the lectin domain and αv integrins via the stalk region. The RKC motif is also distinct from the sequence in the stalk region reported to bind to MHC class II molecules⁵¹. The proteolytic processing pathways that generate soluble CD23 proteins are well understood. Importantly, the RKC motif is preserved in all forms of sCD23 that possess cytokine activity, including the 16 kDa form.

The absence of αvβ5 from peripheral B cells indicates the integrin has no role as a CD23 receptor in these cells, and that its function(s) is limited to the interaction of B cell precursors with bone marrow stroma. The expression of αvβ5 on a subset of CD19⁺/CD9⁺/CD10⁺ bone marrow-derived cells, and on B cell ALL cells (including CD10⁻ leukaemias), indicates that αvβ5 is expressed from a very early stage in B lymphopoiesis. The large population of αvβ5⁺ non-adherent marrow cells that is CD19⁻ is likely to include precursors of T and myelomonocytic cells, since both T-ALL and AML cells are also αvβ5⁺; thus, expression of αvβ5 may also be functionally important for haematopoietic cells other than B cells. Surprisingly, since elevated plasma levels of sCD23 in B-CLL patients are correlated with poor prognosis⁵², there was no detectable expression of αvβ3 or αvβ5 in any B-CLL sample. This argues in favour of active silencing of β5 expression in mature B cells and restriction of αvβ5 pro-survival function to precursor cells.

The importance of adhesion interactions between pre-B cells and ALL cells with stromal cells, for survival of lymphoid cells is widely appreciated, and the VLA-4/VCAM-1 interaction is particularly prominent. The data of FIG. 1B indicate that the CD23-αvβ5 interaction is also critical for promoting growth of SMS-SB cells on CD23⁺ stromal cells in vitro. Thus, in the well-described interactions of ALL blasts and pre-B-like cell lines with stromal elements⁵³⁻⁵⁵, in an environment where all αv ligands (Fn, Vn,⁵⁶ and CD23⁵⁷) are present, the availability of ligand-selective responses, mediated via two distinct binding sites on the same integrin, may be important. If engagement of the RGD-binding site was linked to cell survival, then Vn and Fn in the bone marrow would sustain growth of precursor cells in a non-selective manner; this could preclude elimination of precursors with non-productive rearrangements of antigen receptor genes or malignant precursors. However, the αvβ5 integrin may allow B cell precursors to adhere to the stromal matrix via Vn in an interaction that is neutral with respect to cell survival. Signals for inhibition of apoptosis and promotion of cell growth would then be delivered via the second, non-RGD binding site on the αvβ5 integrin. Whether αvβ5 integrin delivers a growth-sustaining signal to cell types other than lymphoid precursors remains to be established.

This study demonstrates a new role for CD23, regulation of human B cell precursor growth, and defines a further CD23 receptor, the αvβ5 integrin, and its point of contact on the CD23 protein. Expression of the integrin on B cell precursors and ALL cells suggests that αvβ5 may have a role in sustaining normal and leukaemic B cell growth. The data demonstrate that survival signalling via the integrin is both ligand-selective and mediated via a structure on the integrin distinct from the RGD-binding site that recognises a basic domain on CD23. This study underscores the complex roles of CD23 in regulating human B cell function and suggests that the distinct biological functions of CD23 are programmed by discrete structural motifs on the protein. A high-resolution structural model of CD23 will be valuable in elucidating the relationships of such structural motifs.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### REFERENCES

1. Gould, H. J. et al. The biology of IgE and the basis of allergic disease. Annu. Rev. Immunol. 21, 579-628 (2003).
2. Bonnefoy, J.-Y. et al. Structure and Functions of CD23. Int. Rev. Immunol. 16, 113-128 (1997).
3. Delespesse, G., Sarfati, M., Wu, C. Y., Fournier, S. & Letellier, M. The low affinity receptor for IgE. Immunological Reviews 125, 77-97 (1992)
4. Fujiwara, H. et al. The absence of IgE antibody-mediated augmentation of immune responses in CD23-deficient mice. Proc. Natl. Acad. Sci. USA 91, 6835-6839 (1994).
5. Yu, P., Kosco-Vilbois, M. H., Richards, M., Kohler, G. & Lamers, M. C. Negative feedback regulation of IgE synthesis by murine CD23. Nature 369, 753-756. (1994).
6. Dierks, S. E. et al. The oligomeric nature of the murine FceRII/CD23: implications for function. J. Immunol. 150, 2372-2382. (1993).
7. Kilmon, M. A. et al. Regulation of IgE production requires oligomerisation of CD23. J. Immunol. 167, 3139-3145 (2001).
8. Bailey, S. et al. Hydroxamate-bsed inhibitors of low affinity IgE receptor (CD23) processing. Bioorg & Med Chem Lett 8, 23-28 (1998).
9. Marolewski, A. E. et al. CD23 (FceRII) release from cell membranes is mediated by a membrane-bound metalloprotease. Biochem. J. 333, 573-579 (1998).
10. Mossalayi, M. D. et al. Cytokine effects of CD23 are mediated by an epitope distinct from the IgE binding site. EMBO Journal 11, 4323-4328 (1992).
11. Beavil, R. L., Graber, P., Aubonney, N., Bonnefoy, J.-Y. & Gould, H. J. CD23/FceRII and its soluble fragments can form oligomers on the cell surface and in solution. Immunology 84, 202-206 (1995).
12. Mossalayi, D. et al. Soluble CD23 (FceRII) and interleukin 1 synergistically induce early human thymocyte maturation. Journal of Experimental Medicine 171, 959-964 (1990).
13. Mossalayi, M. D. et al. Proliferation of early human myeloid precursors induced by interleulin 1 and recombinant soluble CD23. Blood 75, 1924-1927 (1990).
14. Liu, Y.-J. et al. Recombinant 25-kDa CD23 and interleukin-1a promote the survival of germinal centre B cells: evidence for bifurcation in the development of centrocytes rescued from apoptosis. European Journal of Immunology 21, 1107-1114 (1991).
15. White, L. J. et al. Inhibition of apoptosis in a human pre-B cell line by CD23 is mediated via a novel receptor. Blood 90, 234-243 (1997).
16. Lecoanet-Henchoz, S. et al. CD23 regulates monocyte activation through a novel interaction with the adhesion molecules CD11b-CD18 and CD11c-CD18. Immunity 2, 1-20 (1995).
17. Hermann, P. et al. The Vitronectin Receptor and its associated CD47 molecule mediates proinflammatory cytokine synthesis in human monocytes by interaction with soluble CD23. J. Cell. Biol. 144, 767-775. (1999).
18. Mossalayi, M. D., Arock, M. & Debré, P. CD23/FceRII: signalling and clinical implication. Int. Rev. Immunol. 16, 129-146. (1997).
19. Bansal, A. S., Bruce, J., Hogan, P. G., Prichard, P. & Powell, E. E. Serum soluble CD23, but not IL8, IL10, GM-CSF or IFNg is elevated in patients with hepatitis C infection. Clin. Immunol. Immunopathol 84, 139-144 (1997).
20. Bansal, A., Roberts, T., Hay, E. M., Kay, R. & Pumphrey, R. S. Soluble CD23 levels are elevated in the serum of patients with primary Sjögren's syndrome and systemic lupus erythematosus. Clinical & Experimental Immunology 89, 452-455 (1992).
21. Huissoon, A. P., Emery, P., Bacon. P. A., Gordon, J. & Salmon, M. Increased expression of CD23 in rheumatoid synovitis. Scand. J. Rheumatol. 29, 154-159. (2000).
22. Bansal, A. S. et al. Increased levels of sCD23 in rheumatoid arthritis are related to disease status. Clinical & Experimental Rheumatology 12, 281-285 (1994).
23. Sarfati, M. et al. Prognostic importance of serum soluble CD23 level in chronic lymphocytic leukaemia. Blood 88, 4259-4264 (1996).
24. Aubry, J.-P., Pochon, S., Graber, P., Jansen, K. U. & Bonnefoy, J.-Y. CD21 is a ligand for CD23 and regulates IgE production. Nature 358, 505-507 (1992).
25. Bjorck, P., Elenstrom-Magnusson, C., Rosen, A., Severinson, E. & Paulie, S. CD23 and CD21 function as adhesion molecules in homotypic aggregation of human B lymphocytes. European Journal of Immunology 23, 1771-1775 (1993).
26. Bonnefoy, J.-Y., Henchoz, S., Hardie, D., Holder, M. J. & Gordon, J. A subset of anti-CD21 antibodies promote the rescue of germinal centre B cells from apoptosis. European Journal of Immunology 23, 969-972 (1993).
27. Aubry, J.-P. et al. CD23 interacts with a new functional extracytoplasmic domain involving N-linked oligosaccharides on CD21. Journal of Immunology 152, 5806-5813 (1994).
28. Lecoanet-Henchoz, S. et al. Mouse CD23 regulates monocyte activation through an interaction with the adhesion molecular CD11b/CD18. Eur J Immunol 27, 2290-2294. (1997).
29. van der Flier, A. & Sonnenberg, A. Function and Interactions of integrins. Cell Tissue Res 305, 285-298 (2001).
30. Marshall, J. F. & Hart, I. R. The role of aᵥ integrins in tumour progression and metastasis. Semin. Cancer Biol. 7, 129-138 (1996).
31. Kumar, C. C. Signaling by integrin receptors. Oncogene 17, 1365-1373. (1998).
32. Hood, J. D. & Cheresh, D. A. Role of integrins in cell invasion and migration. Nature Reviews Cancer 2, 91-100 (2002).
33. Stupack, D. W. & Cheresh, D. A. Get a ligand, get a life: integrins, signaling and cell survival. J. Cell. Sci. 115, 3729-3738 (2002).
34. Finnemann, S. C. & Rodriguez-Boulan, E. Macrophage and retinal pigment epithelium phagocytosis: apoptotic cells and photoreceptors compete for avb3 and avb5 integrins, and protein kinase C regulates avb5 binding and cytoskeletal linkage. J. Exp. Med 190, 861-874. (1999).
35. Ruoslahti, E. RGD and other recognition sequences for integrins. Annu. Rev. Cell. Biol. 12, 697-715. (1996).
36. Xiong, J.-P. et al. Crystal structure of the extracellular segment of integrin avb3. Science 294, 339-345. (2001).
37. Xiong, J.-P. et al. Crystal structure of the extracellular segment of integrin avb3 in complex with an arg-gly-asp ligand. Science 296, 151-155 (2002).
38. Vogel, B. E. et al. A novel integrin specificity exemplified by binding of the avb5 integrin to the basic domain of the HIV Tat protein and vitronectin. J. Cell. Biol 121, 461-468. (1993).
39. Smith, R. G. Paraosteal Lymphoblastic Lymphoma: Ahuman counter part of Abelson Virus-induced lymphosarcoma of mice. Cancer 54, 471-476 (1984).
40. Simonsen, J. L. et al. Telomerase expression extends the proliferative lifespan and maintains the osteogenic potential of human bone marrow stromal cells. Nat. Biotechnol. 20, 560-561 (2002).

41. Suzuki, S. et al. cDNA and amino acid sequences of the cell adhesion receptor recognising vitronectin reveal a transmembrane domain and homologies to other adhesion protein receptors. Proc. Natl. Acad. Sci. USA 83, 8614-8618 (1986).
42. Wayner, E. A., Orlando, R. A. & Cheresh, D. A. Integrins avb3 and avb5 contribute to cell attachment to vitronectin but differentially distribute on the cell surface. J. Cell. Biol 113, 919-929 (1991).
43. Cheresh, D. A. Human endothelial cells synthesize and express an Arg-Gly-Asp-directed adhesion receptor involved in attachment to fibrinogen and von Willebrand factor. Proc. Natl. Acad. Sci. USA 84, 6471-6475. (1987).
44. Stuiver, I. & Smith, J. W. Characterisation of monoclonal antibodies against integrin alpha V beta 5. Hybridoma 14, 545-550 (1995).
45. Butler, B., Williams, M. P. & Blystone, S. D. Ligand-dependent activation of integrin avb3. J. Biol. Chem. 278, 5264 (2003).
46. Ivaska, J. et al. A epetide inhibiting collagen binding function of integrin a2I domain. J. Biol. Chem. 274, 3513-3521. (1999).
47. Pentikainen, O. et al. “RKKH” peptides from the snake venom metalloprotease of Bothrops jararaca bind near the metal ion-dependent adhesion site of the human integrin a2 I-domain. J. Biol. Chem. 274, 31493-31505. (1999).
48. Nymalm, Y. et al. Jararhagin-derived RKKH peptides induce strucural changes in a1I domain of human integrin a1b1. J. Biol. Chem. 279, 7962-7970. (2004).
49. Padlan, E. A. & Helm, B. A. Modelling of the lectin homology domains of the human and murine low affinity Fce receptor (FceRII/CD23). Receptor 3, 325-341 (1993).
50. Bajorath, J. & Aruffo, A. Structure-based modelling of the ligand binding domain of the human cell surface receptor CD23 and comparison of two independently derived moelcular models. Protein Sci. 5, 240-247. (1996).
51. Kijimoto, O., S., & Noguchi, A. Two peptides from CD23, including the inverse RGD sequence and its related peptide, interact with the MHC class II Molecule. Biochem. Biophys. Res. Commun. 267, 686-691. (2000).
52. Sarfati, M. et al. Prognostic importance of serum soluble CD23 level in chronic lymphocytic leukaemia. Blood 88, 4259-4264 (1996).
53. Hewson, J., Bianchi, A., Bradstock, K., Makrynikola, V. & Gottlieb, D. Ultrastructural changes during adhesion and migration of pre-B lymphoid leukaemia cells within bone marrow stroma. Br J Haematol 92, 77-87. (1996).
54. Makrynikola, V., Bianchi, A., Bradstock, K., Gottlieb, D. & Hewson, J. Migration of acute lymphoblastic leukemia cells into human bone marrow stroma. Leukemia 8, 1734-43. (1994).
55. Makrynikola, V. & Bradstock, K. F. Adhesion of precursor-B acute lymphoblastic leukaemia cells to bone marrow stromal proteins. Leukemia 7, 86-92. (1993).
56. Gronthos, S., Simmons, P. J., Graves, S. E. & Robey, P. G. Integrin-mediated interactions between human bone marrow stromal precursor cells and the extracellular matrix. Bone 28, 174-81. (2001).
57. Fourcade, C. et al. Expression of CD23 by human bone marrow stromal cells. European Cytokine Network 3, 539-543. (1992).
58. Chamberlain, J. P. Analyt. Biochem. 98, 132-135 (1979).

1. A method of inhibiting the interaction between an αv integrin and CD23, the method comprising contacting the αv integrin with a peptide capable of binding to αvβ5, the peptide comprising the motif X1X2X3, wherein at least one of X1, X2 and X3 is a residue carrying at least a partial positive charge at physiological pH, and one of X1 and X3 is C.

2. A method according to claim 1 wherein the αv integrin is cell-associated or immobilised on a solid phase and said CD23 is optionally cell-associated or immobilized on a solid phase.

3. (canceled)

4. A method according to claim 1 wherein the αv integrin is selected from the group consisting of αvβ3, αvβ5, αvβδ and αvβ8.

5. The method of claim 1, wherein said inhibiting results in reduced CD23-dependent proliferation or survival of a cell.

6. A method according to claim 5 wherein the cell is a pre-B cell or a cancer cell.

7. A method according to claim 6 wherein the cancer cell is an ALL cell or myeloma cell.

8. A method according to claim 7 wherein the ALL cell is from the B cell lineage.

9. A method of treating cancer or inflammatory disorders in a subject, comprising administering to the subject an effective amount of (i) a peptide capable of binding to αvβ5, the peptide comprising the motif X1X2X3, wherein at least one of X1, X2 and X3 is a residue carrying at least a partial positive charge at physiological pH, and one of X1 and X3 is C, or (ii) a nucleic acid encoding said peptide.

10. (canceled)

11. A method of inhibiting cytokine secretion from a monocytic cell comprising contacting the cell with a peptide capable of binding to αvβ5, the peptide comprising the motif X1X2X3, wherein at least one of X1, X2 and X3 is a residue carrying at least a partial positive charge at physiological pH, and one of X1 and X3 is C, wherein said cell is optionally a monocyte or a macrophage.

12. A method according to claim 11 wherein the cell is a monocyte or macrophage.

13. A method according to claim 11 wherein the cytokine is a pro-inflammatory cytokine such as TNF-α, IL-1, IL-6, IL-8, IL-12 or IFN-γ.

14. (canceled)

15. A method according to claim 9 wherein the inflammatory disorder is rheumatoid arthritis, Sjogren's Syndrome, systemic lupus erythaematosus, endometriosis, sarcoidosis, thyroiditis or atherosclerosis.

16. (canceled)

17. A method of screening for a substance capable of inhibiting the interaction between an αv integrin and CD23, the method comprising
(i) contacting the αv integrin with a test substance,
(ii) contacting the αv integrin with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(iii) determining binding of the peptide to the αv integrin, said method optionally comprising the step of selecting or rejecting the test substance depending on its effect on peptide binding to the αv integrin, isolated integrin β chain or isolated extracellular domain.

18. A method of screening for a substance capable of inhibiting the interaction between an αv integrin and CD23, the method comprising
(i) contacting an isolated integrin β chain or isolated extracellular domain thereof with a test substance,
(ii) contacting the isolated integrin β chain or isolated extracellular domain with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(iii) determining binding of the peptide to the isolated integrin β chain or isolated extracellular domain, said method optionally comprising the step of selecting or rejecting the substance depending on its effect on peptide binding to the αv integrin, isolated integrin β chain or isolated extracellular domain.

19. A method according to claim 18 wherein the isolated integrin β chain or isolated extracellular domain is β3, 5, 6 or 8.

20. A method according to claim 17 wherein the αv integrin is cell-associated or immobilised on a solid phase.

21. A method according to claim 18 wherein the isolated integrin β chain or isolated extracellular domain is immobilised on a solid phase.

22. (canceled)

23. A method according to claim 17 wherein the peptide is labelled.

24. A method according to claim 17 comprising the step of contacting a cell expressing the αv integrin with said test substance and determining the effect of the test substance on the cell.

25. A method according to claim 24 comprising determining whether the test substance affects at least one of the following
i) the apoptotic state of the cell,
ii) cellular proliferation, and
iii) cytokine expression and/or secretion by the cell, optionally in response to a suitable stimulus.

26-27. (canceled)

28. The method of claim 25, wherein said cell is a cancer cell

29. The method of screening as claimed in claim 25, wherein said cell is a monocytic cell which is contacted with said test substance and determining the effect of the substance on inflammatory cytokine expression and/or secretion by the cell is determined.

30. A method according to claim 29 comprising administering a suitable stimulus to the cell which, in the absence of the test substance, provokes inflammatory cytokine expression and/or secretion.

31. A method according to claim 25 comprising determining expression and/or secretion of TNF-α, IL-I, IL-6, IL-8, IL-12 or IFN-γ.

32. A method according to claim 25 comprising administering said peptide to an animal model for said cancer or inflammatory disease.

33. A method according to claim 32 wherein the animal model is a rodent capable of expressing a CD23 protein having R at a position corresponding to R172 of human CD23.

34. A method of stimulating proliferation or inhibiting apoptosis of a cell expressing an αv integrin, the method comprising contacting the cell with an αvβ5 agonist peptide, the peptide comprising the motif X1X2X3, wherein at least one of X1, X2 and X3 is a residue carrying at least a partial positive charge at physiological pH, and one of X1 and X3 is C, wherein said cell is optionally a pre-B cell.

35. A method according to claim 34 wherein the cell expresses αvβ5.

36. (canceled)

37. A method of determining expression of an αv integrin by a cell, the method comprising:
(i) contacting the cell with a peptide capable of binding to αv′5, the peptide comprising the motif X1X2X3, wherein at least one of X1, X2 and X3 is a residue carrying at least a partial positive charge at physiological pH, and one of X1 and X3 is C, and
(ii) determining binding of said peptide to said cell, wherein said peptide is optionally labelled.

38. A method according to claim 37 comprising correlating the result with the level of expression of αv.

39. (canceled)

40. A method of screening for the presence of an ALL cell in a sample comprising blood cells, the method comprising
(i) contacting the sample with a peptide capable of binding to αvβ5, the peptide comprising the motif X1X2X3, wherein at least one of X1, X2 and X3 is a residue carrying at least a partial positive charge at physiological pH, and one of X1 and X3 is C, and
(ii) determining binding of said peptide to said blood cells.

41. A method according to claim 40 further comprising contacting the sample with a B cell-specific binding agent.

42. A method according to claim 41 wherein the B cell-specific binding agent is labelled with a different label to that carried by the peptide.

43. A method of isolating an αv integrin from a sample, which is optionally a cell lysate, comprising contacting said sample with a peptide capable of binding to αvβ5, the peptide comprising the motif X1X2X3 wherein at least one of X1, X2 and X3 is a residue carrying at least a partial positive charge at physiological pH, and one of X1 and X3 is C.

44. A method according to claim 43 wherein the motif X1X2X3 of the peptide capable of binding to αvβ5 comprises only one C residue.

45. A method according to claim 44 wherein the residue carrying at least a partial positive charge at physiological pH is K, R or H.

46. A method according to claim 45 wherein any of X1, X2 and X3 which is neither C nor a residue carrying at least a partial positive charge is a neutral residue.

47. A method according to claim 46 wherein the neutral residue is Q, N, A, G, S, T, V, L or I.

48. A method according to claim 47 wherein the neutral residue is A, Q, S or G.

49. A method according to claim 44 wherein one of X1 or X3 is C, the other of X1 and X3 is Q, R, K, H, A, G or S, and X2 is K, R, Q, H, A, G or S, provided that one of X1, X2 and X3 is K, R or H.

50. A method according to claim 49 wherein one of X1, X2 and X3 is K or R.

51. A method according to claim 49 wherein one of X1 and X3 is C, the other of X1 and X3 is Q, R or A, and X2 is K or A, provided that one of X1, X2 and X3 is K or R.

52. A method according to claim 44 wherein peptide comprises the motif XaX1X2X3 or X1X2X3Xa, wherein Xa is Q, K or R.

53. A method according to claim 52 wherein the motif X1X2X3 has the sequence RKC, QKC, AKC, RAC or CKR.

54. A method according to claim 44 wherein peptide comprises one or more repeats of a CD23 sequence of up to 20 amino acids, the CD23 sequence having at least 70%, sequence identity with the corresponding portion of amino acids 155 to 191 of SEQ ID NO: 1.

55. A method according to claim 54 wherein the peptide comprises the sequence QRKC, FQRKC, NFQRKC, INFQRKC, WINFQRKC, KWINFQRKC, RKCY, RKCYY, RKCYYF, RKCYYFG, RKCYYFGK, RKCYYFGKG, QRKCYY, FQRKCYY, FQRKCYYF, FQRKCYYFG, NFQRKCY, INFQRKCY, INFQRKCYY, or an equivalent sequence from a non-human CD23 protein.

56. A method according to claim 1 wherein the αv integrin is a rodent integrin and the peptide comprises the motif X1X2X3, wherein X3 is C, and X1 is a residue carrying at least a partial positive charge at physiological pH.

57. A method according to claim 56 wherein X1 is R or K, and wherein X2 is optionally a residue carrying at least a partial positive charge at physiological pH.

58. (canceled)

59. A method according to claim 57 wherein X2 is K or R.

60. A method according to claim 56 wherein the peptide comprises the sequence:
QRKC,
FQRKC,
HFQRKC,
LHFQRKC,
WLHFQRKC,
NWLHFQRKC,
RKCY,
RKCYY,
RKCYYF,
RKCYYFG,
RKCYYFGK,
RKCYYFGKG,
QRKCYY,
FQRKCYY,
FQRKCYYF,
FQRKCYYFG,
HFQRKCY,
LHFQRKCY,
or
WLHFQRKCYY.

61. A method according to claim 56 wherein the peptide comprises the sequence: DWLHFQRKC, RKCYYFGE, or RKCYYFGEG.

62. A method according to claim 60 wherein the peptide is cyclic.

63. A peptide comprising a CD23 sequence of up to but not exceeding 20 amino acids, the CD23 sequence comprising the motif X1X2X3, wherein at least one of X1, X2 and X3 is a residue carrying at least a partial positive charge at physiological pH, and one of X1 and X3 is C, the CD23 sequence further having at least 80% sequence identity with the corresponding portion of amino acids 155 to 191 of SEQ ID NO: 1.

64. A peptide according to claim 63 wherein the CD23 sequence comprises one of the sequences QRKC, FQRKC, NFQRKC, INFQRKC, WINFQRKC, KWINFQRKC, RKCY, RKCYY, RKCYYF, RKCYYFG, RKCYYFGK, RKCYYFGKG, QRKCYY, FQRKCYY, FQRKCYYF, FQRKCYYFG, NFQRKCY, INFQRKCY, INFQRKCYY, or the equivalent sequence from a non-human CD23 molecule.

65. A nucleic acid encoding a peptide according to claim 63.

66. (canceled)

67. A pharmaceutical composition comprising a peptide according to claim 63, or a nucleic acid encoding said peptide, in combination with a pharmaceutically acceptable excipient.

68. A rodent cell genetically modified to contain nucleic acid encoding a CD23 protein having R at a position corresponding to R172 of human CD23, said rodent cell optionally being a mouse or a rat cell and said CD23 protein optionally being human in origin.

69. A rodent cell according to claim 68 wherein the CD23 protein has the motif RKC at positions corresponding to positions 172 to 174 of human CD23.

70-71. (canceled)

72. A rodent cell according to claim 68 wherein the endogenous chromosomal CD23 gene has been modified to encode a CD23 protein having R at the position corresponding to R172 of human CD23.

73. A rodent cell according to claim 68 wherein the nucleic acid encoding the CD23 protein is extrachromosomal.

74. A rodent cell according to claim 68 wherein the nucleic acid encoding the CD23 protein is stably integrated into a chromosome of the cell.

75. A rodent cell according to claim 68 wherein the cell does not express endogenous CD23.

76. A transgenic rodent selected from the group consisting of rodents which comprise a nucleic acid encoding CD23 stably integrated into cells of said rodent and rodents which do not express endogenous CD23.

77. A method of testing a substance for a prophylactic or therapeutic effect on an inflammatory disorder, comprising administering said test substance to a transgenic rodent according to claim 76, said transgenic rodent being affected by, or suspected of being likely to develop, said inflammatory disorder.

78. A method according to claim 77 wherein the transgenic rodent has a genetic background suitable for use as a model for rheumatoid arthritis, Sjogren's Syndrome, systemic lupus erythaematosus (SLE), sarcoidosis, endometriosis, thyroiditis or atherosclerosis.

79. A method of testing a substance for a prophylactic or therapeutic effect on a neoplastic disorder, comprising administering said test substance to a transgenic rodent according to claim 76, said transgenic rodent being affected by, or suspected of being likely to develop, said neoplastic disorder.

80. A method according to claim 79 wherein said neoplastic disorder is a pre-B cell cancer or a myeloma.

81. A mutant of a wild type CD23 protein, wherein the wild type protein has a Q at a position corresponding to R172 of human CD23, the mutant comprising a residue having at least a partial positive charge at physiological pH at the position corresponding to R172 of human CD23.

82. A mutant protein according to claim 81 wherein the mutant has R or K at the position corresponding to R172 of human CD23.

83. An isolated extracellular domain of a mutant protein according to claim 81.

84. An isolated peptide comprising at least 15 amino acids from a mutant protein according to claim 81, said peptide comprising the motif RKC at the positions corresponding to 172 to 174 of human CD23.

85. An isolated nucleic acid comprising an open reading frame encoding a mutant protein, extracellular domain or peptide according to claims 81.

86. An expression vector comprising an isolated nucleic acid according to claim 85, optionally contained within a host cell.

87. (canceled)
